(19) United States
(12) Patent Application Publication
(10) Pub. No.: US 2026/0331231 A1
(43) Pub. Date: Oct. 22, 2026

(54) SYSTEMS AND METHODS FOR NEW INTERNAL DONOR DISCOVERY

(71) Applicant: W.R. Grace & Co.-CONN., Columbia, MD (US)

(72) Inventors: Joseph N. Coalter, III, Hurricane, WV (US); Julie L. Boyer, Columbia, MD (US); Nathan Eberhardt, Columbia, MD (US)

(73) Assignee: W.R. Grace & Co.-CONN., Columbia, MD (US)

(21) Appl. No.: 19/182,507

(22) Filed: Apr. 17, 2025

(51) Int. Cl. G16C 20/40; G06Q 50/04; G16C 20/70

(52) U.S. Cl. CPC G16C 20/40; G06Q 50/04; G16C 20/70

(57) ABSTRACT

The present disclosure is directed to systems and methods to identify a data structure comprising one or more chemical structures as candidate internal donors for polyolefin procatalysts, the one or more chemical structures represented as a data string defined according to a predetermined specification; provide the data structure as input to a machine-learning model to generate a predicted internal donor percentage for each resulting polyolefin procatalyst created using each chemical structure of the one or more chemical structures in the data structure; and provide, for presentation, an output including the predicted internal donor percentage for each of the one or more chemical structures for the internal donor. The model is updated using a plurality of training examples, each of the plurality of training examples comprises a respective chemical structure for the internal donor, and, optionally, a respective internal donor incorporation in polyolefin procatalyst percentage corresponding to the respective chemical structure.

### BACKGROUND

Ziegler-Natta (Z/N) type catalysts are used in the production of olefin polymers. Generally, Ziegler-Natta catalysts for polypropylene may include a magnesium compound-supported titanium compound, and an organic molecule known as an internal donor. This procatalyst is combined with aluminum compound co-catalyst, and optionally an external electron donor in the presence of alpha olefin in the polymerization reactor to afford polypropylene.

### SUMMARY

Ziegler-Natta catalyst compositions can optionally be made using one or more internal electron donors. The internal electron donors are bound to the procatalyst and added during the catalyst synthesis process. The internal electron donors can determine to a large extent the performance properties of the overall catalyst composition, such as the catalyst activity and catalyst lifetime. The internal electron donors can also influence the properties of polymers made from the catalyst composition. For instance, internal electron donors can influence polymer melt flow rates, xylene soluble content, molecular weight distribution and the like.

Conventional phthalate internal donors have certain disadvantages, which may include heightened regulatory scrutiny, restricted uses, and issues with catalyst performance that lead to undesirable polymer properties (e.g., xylene solubility, melt flow index, and molecular weight distribution).

At least one aspect of the present disclosure is directed to a system. The system may include one or more processors coupled to non-transitory memory. The one or more processors may be configured to identify a data structure comprising one or more chemical structures as candidate internal donors for polyolefin procatalysts, the one or more chemical structures represented as a data string defined according to a predetermined specification. The one or more processors may be configured to provide the data structure as input to a machine-learning model to generate a predicted internal donor percentage for each resulting polyolefin procatalyst created using each chemical structure of the one or more chemical structures in the data structure. The machine-learning model may be updated using a plurality of training examples, each of the plurality of training examples including (i) a respective chemical structure for the internal donor, and, optionally, (ii) a respective internal donor incorporation in polyolefin procatalyst percentage corresponding to the respective chemical structure. The one or more processors may be configured to provide, for presentation, an output including the predicted internal donor percentage in the procatalyst composition for each of the one or more chemical structures for the internal donor.

In some implementations, the one or more processors may be further configured to update the machine-learning model using the plurality of training examples. Each of the plurality of training examples may further comprise one or more polymerization parameters and one or more polyolefin properties. The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value. Each of the plurality of training examples may further include one or more of (i) a set of calculated physical properties having at least one of an electronegativity value, a hardness value, or a dipole moment, and (ii) a set of calculated three-dimensional (3D) descriptors.

In some implementations, the one or more processors may be further configured to generate a feature importance for one or more values of a dataset, and generate the plurality of training examples based on the feature importance. The one or more processors may be further configured to generate, for each chemical structure of the one or more chemical structures, a respective set of molecular descriptors based on the data string of the chemical structure; and provide the respective set of molecular descriptors as input to the machine-learning model. The one or more processors may be further configured to provide the data structure as input to using at least one application programming interface (API) call. The one or more processors may be further configured to generate, using the machine-learning model for each chemical structure of the one or more chemical structures, a confidence score indicating a likelihood that the predicted internal donor percentage in the procatalyst composition for the chemical structure is accurate. The one or more processors may be further configured to generate one or more plots based on the output of the machine-learning model.

At least one aspect of the present disclosure is directed to a method. The method may include receiving, by one or more processors coupled to non-transitory memory, a data structure comprising one or more chemical structures as candidate internal donors in polyolefin procatalyst, the one or more chemical structures represented as a data string defined according to a predetermined specification. The method may include providing, by the one or more processors, the data structure as input to a machine-learning model to generate a predicted internal donor percentage in the procatalyst composition for each resulting polyolefin procatalyst created using each chemical structure of the one or more chemical structures in the data structure. The machine-learning model may be updated using a plurality of training examples, each of the plurality of training examples comprising: (i) a respective chemical structure, and (ii) a respective internal donor percentage in the procatalyst composition corresponding to the respective chemical structure. The method may include providing, by the one or more processors, for presentation an output including the predicted internal donor percentage in the procatalyst composition for each of the one or more chemical structures.

In some implementations, the method may include updating, by the one or more processors, the machine-learning model using the plurality of training examples. Each of the plurality of training examples may include one or more polymerization parameters and one or more polyolefin properties. The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or a melt flow response (MFR) value. Each of the plurality of training examples may include one or more of a set of calculated physical properties, a set of calculated three-dimensional (3D) descriptors, an electronegativity value, a hardness value, or a dipole moment.

In some implementations, the method may include generating, by the one or more processors, a feature importance for one or more values of a dataset; and generating, by the one or more processors, the plurality of training examples based on the feature importance. The method may include generating, by the one or more processors, for each chemical structure of the one or more chemical structures, a respective set of molecular descriptors based on the data string of the chemical structure; and providing, by the one or more processors, the respective set of molecular descriptors as input to the machine-learning model.

At least one aspect of the present disclosure is directed to a system. The system may include one or more processors coupled to non-transitory memory. The one or more processors may be configured to identify a data structure comprising one or more chemical structures as candidate internal donors for polyolefin procatalysts, the one or more chemical structures represented as a data string defined according to a predetermined specification. The one or more processors may be configured to provide the data structure as input to a machine-learning model to generate a predicted polymer performance and a predicted catalyst performance for each resulting polyolefin procatalyst created using each chemical structure of the one or more chemical structures in the data structure. The machine-learning model may be updated using a plurality of training examples, each of the plurality of training examples comprising (i) a respective chemical structure, (ii) a respective catalyst performance, and (iii) one or more respective polymer properties corresponding to the respective chemical structure. The one or more processors may be configured to provide, for presentation, an output including the predicted polymer performance and a predicted catalyst performance for each of the one or more chemical structures.

In some implementations, the respective catalyst performance may include one or more of a catalytic activity value or a catalyst lifetime value, and wherein the one or more respective polymer properties comprise one or more of a xylene solubility (XS) value, a polydispersity index (PDI) value, or a melt flow response (MFR) value. The one or more processors may be further configured to generate, using the machine-learning model for each chemical structure of the one or more chemical structures, one or more confidence scores indicating a likelihood that the predicted polymer performance and the predicted catalyst performance are accurate.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the present disclosure can be combined with other aspects. Aspects can be implemented in any convenient form. For example, by appropriate computer programs, which may be carried on appropriate carrier media (computer readable media), which may be tangible carrier media (e.g., disks) or intangible carrier media (e.g., communications signals). Aspects may also be implemented using suitable apparatus, which may take the form of programmable computers running computer programs arranged to implement the aspect.

### BRIEF DESCRIPTION OF THE DRAWING

The accompanying drawings are not intended to be drawn to scale. Like reference numbers and designations in the various drawings indicate like elements. For purposes of clarity, not every component may be labeled in every drawing. In the drawings:

FIG. 1 illustrates an example system for predicting polyolefin procatalyst internal donor percentage in the procatalyst composition using a machine learning model updated using a plurality of training examples, each training example including chemical structure and respective internal donor percentage.

FIG. 2 illustrates an example system for predicting polyolefin procatalyst internal donor percentage in the procatalyst composition using a machine learning model updated using a plurality of training examples, each training example including chemical structure, respective internal donor percentage, polymerization parameter, and polyolefin property.

FIG. 3 illustrates an example system for predicting polyolefin procatalyst internal donor percentage in the procatalyst composition using a machine learning model updated using a plurality of training examples, each training example including chemical structure, respective internal donor percentage, polymerization parameter, polyolefin property, physical properties, and 3D descriptor.

FIG. 4 illustrates an example system for predicting polyolefin procatalyst internal donor percentage in the procatalyst composition using a feature importance generator and using a machine learning model updated using a plurality of training examples, each training example including chemical structure and respective internal donor percentage.

FIG. 5 illustrates an example system for predicting polyolefin procatalyst internal donor percentage in the procatalyst composition using molecular descriptors of candidate internal donors and a machine learning model updated using a plurality of training examples, each training example including chemical structure and respective internal donor percentage.

FIG. 6 illustrates an example system for predicting polyolefin procatalyst internal donor percentage in the procatalyst composition using an application programming interface (API) and a machine learning model updated using a plurality of training examples, each training example including chemical structure and respective internal donor percentage.

FIG. 7 illustrates an example system for identifying a data structure including one or more candidate internal donors for polyolefin procatalysts and predicting polyolefin procatalyst internal donor percentage using the candidate internal donors and a machine learning model updated using a plurality of training examples, each training example including chemical structure, respective catalyst performance, and respective polymer properties.

FIG. 8 illustrates a flow chart of an example method for predicting polyolefin procatalyst internal donor percentage.

FIG. 9 illustrates a block diagram of an example computing system suitable for use in the various arrangements described herein, in accordance with one or more implementations.

FIG. 10 is a graph of actual vs. predicted internal donor percentage in the procatalyst composition for respective candidate internal donors generated using the system of FIG. 1.

FIG. 11 is a graph of internal donor percentage in the procatalyst composition (“% donor”) vs. catalyst activity (kg/g) for candidate internal donors generated using the system of FIG. 1.

FIG. 12 is a graph of internal donor percentage in the procatalyst composition vs. octanol/water partition coefficient (“XLogP”) for candidate internal donors generated using the system of FIG. 1.

FIGS. 13A-13C are graphs of actual vs. predicted parameters for respective candidate internal donors generated using the system of FIG. 2. FIG. 13A is a graph of actual vs. predicted xylene solubility (XS). FIG. 13B is a graph of actual vs. predicted catalytic activity. FIG. 13C is a graph of actual vs. predicted internal donor percentage in the procatalyst composition.

FIGS. 14A-14C are graphs of predicted vs. actual parameters for respective candidate internal donors generated using the system of FIG. 3. FIG. 14A is a graph of actual vs. predicted catalytic activity. FIG. 14B is a graph of actual vs. predicted xylene solubility. FIG. 14C is a graph of actual vs. predicted internal donor percentage.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

New nonphthalate internal donors are useful for meeting regulatory standards and achieving catalyst performance targets for polymer properties. However, developing new internal donors can be a costly and time-consuming process.

Disclosed herein are systems and methods for identifying candidate internal donors for polyolefin procatalysts using one or more machine-learning models to predict internal donor parameters, including catalyst performance and resulting polymer properties. The procatalyst may include a magnesium compound-supported titanium compound, and an organic molecule known as an internal donor. This procatalyst is combined with aluminum compound co-catalyst, and optionally an external electron donor in the presence of alpha olefin in the polymerization reactor to afford polypropylene.

Conventional approaches for predicting internal donor performance typically provide inconsistent or inaccurate results. This can be due to the fact that larger data sets (e.g., including hundreds of internal donor parameters) are challenging to efficiently and accurately process using conventional approaches. To address these challenges, the systems and methods of this technical solution implement approaches for training and using one or more machine-learning models to predict internal donor performance given a subset of information. The technical solution can involve the use of datasets that omit data that may negatively skew machine-learning predictions. Therefore, the systems and methods disclosed herein may be trained with using datasets including a smaller subset of internal donor parameters (e.g., 1-100, 1-50, 1-20, 1-10, 2-10, or 2-6 parameters). The use of smaller datasets both improves the accuracy of the output data and improves computational performance by reducing the amount of information that is to be processed to generate predictions.

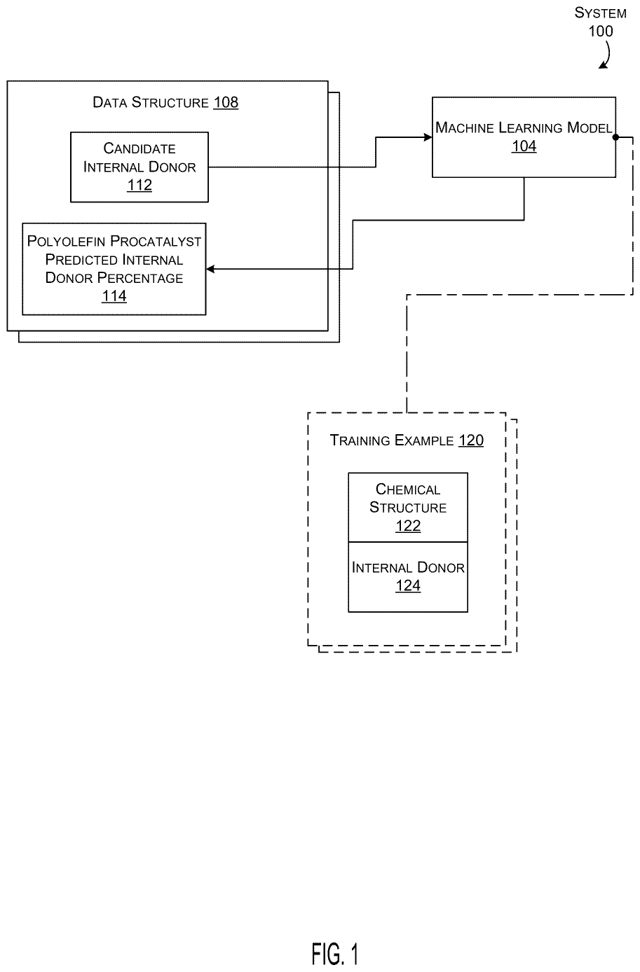

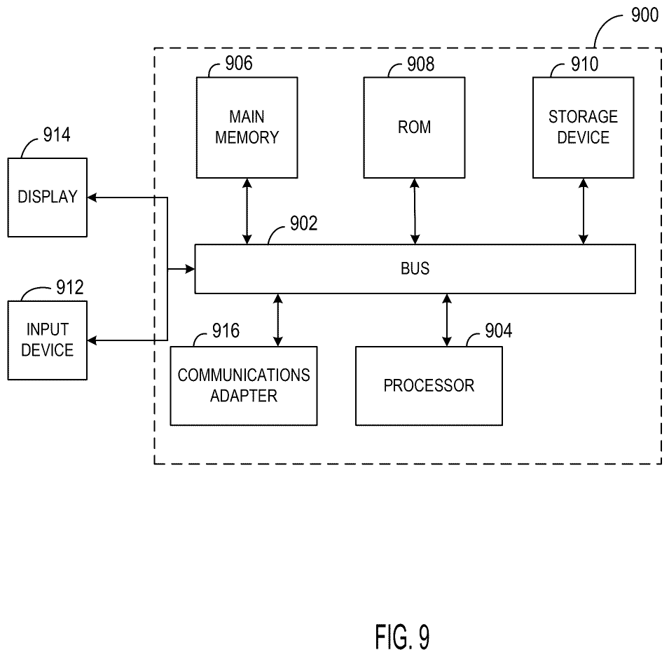

Referring to FIG. 1, illustrated is a block diagram of an example system 100 for predicting polyolefin procatalyst internal donor percentage using a machine learning model updated using a plurality of training examples, each training example including chemical structure and respective internal donor percentage. Operations described in connection with FIG. 1 may be performed via one or more computing devices. For example, the computer system 900 of FIG. 9 may implement some or all of the operations described herein, through the execution of computer-readable instructions.

The system 100 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 100 may include one or more computing devices or servers that can perform various functions as described herein.

The system 100 may execute one or more machine learning models 104 in response to corresponding request(s) from an operator of the system 100 or from an external computing system. Outputs from the system 100 may be displayed on a display device of a computing device executing the system 100. For example, the computer system 900 of FIG. 9 may output predicted percent donor values and may display these values on display 914.

The system 100 may include one or more data structures 108, which may be stored in a storage device (e.g., storage device 910 of computer system 900 in FIG. 9), such as a solid-state device, magnetic disk, or optical disk for persistently storing information and instructions. The data structures 108 may be received from the storage device by a processor (e.g., processor 904 of computer system 900 in FIG. 9) via a bus (e.g., bus 902 of computer system 900 in FIG. 9).

The system 100 may include one or more machine learning models 104, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 100 may include one or more training examples 120, which may be stored in a storage device (e.g., storage device 910 of computer system 900 in FIG. 9), such as a solid-state device, magnetic disk, or optical disk for persistently storing information and instructions. The storage device in which the training examples 120 are stored may be the same or different than the storage device for storing the data structures 108. The training examples 120 may be received from the storage device by the processor (e.g., processor 904 of computer system 900 in FIG. 9) via the bus (e.g., bus 902 of computer system 900 in FIG. 9).

In some implementations, components of the system 100 may be implemented using multiple computing systems in communication with one other, for example, via a network. In some implementations, the system 100 may be implemented at least in part of a distributed computing system For example, the system 100 may be or may include an application server or webserver, which may include software modules to access or transmit data structures 108 stored by the memory in the system 100. In some implementations, one or more client devices of the system 100 may transmit data structures 108 to the distributed computing system in response to user input by an operator of the client devices. Such requests may include requests to initiating training and/or inference operations based on data submitted to the computing system maintaining the machine-learning model 104.

The system 100 may include a network to facilitate communication between remote components of the system 100 and/or between the system 100 and a remote computing system. The network may include any number of hardwired or wireless connections. Any or all of the computing devices described herein (e.g., the system 100) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 100) may also communicate wirelessly with other computing devices via a proxy device (e.g., a router, network switch, or gateway).

The system 100 may identify a data structure 108 comprising one or more chemical structures as candidate internal donors 112 for polyolefin procatalysts. The system may provide the data structure 108 as input to a model 104 to generate a predicted internal donor percentage for each resulting polyolefin procatalyst created using each chemical structure of the one or more chemical structures in the data structure 108. The system 100 may provide, for presentation, an output 114 including the predicted internal donor percentage for each of the one or more chemical structures for the internal donor.

The model 104 may be effective for operating on or generating data representative of chemical structures. The data representative of chemical structures may be data strings defined according to a predetermined specification. For example, the candidate internal donor 112 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 100 includes one or more models 104. The models 104 may be trained using one or more training examples. The model 104 may include machine learning models or other models that can generate target outputs based on various types of inputs.

The system 100 may train, update, and/or configure one or more models 104. The model 104 may include machine learning models or other models that may generate target outputs based on various types of inputs. The system 100 may train, update, and/or configure the model 104 by modifying or updating one or more parameters responsive to evaluating one or more training examples 120 in view of expected or ground truth outputs.

The system 100 may operate on one or more candidate internal donors 112 (e.g., inference or runtime data; training data), which may be retrieved from one or more data structures 108. The data structure may include various databases, molecule editors, sensor data streams, user input sources, or a combination of any two or more thereof from which the system 100 can retrieve candidate internal donor 112 to process.

The data structure 108 may be maintained by one or more entities, which may be entities that maintain the system 100 and/or may be separate from entities that maintain the system 100. In some implementations, the system 100 uses data from different data sets, such as by using candidate internal donors 112 from a first data structure 108 to perform at least a first configuring (e.g., updating or training) of the model 104, and uses training data elements from a second data structure 108 to perform at least a second configuring of the models 104. For example, the first data structure 108 may include publicly available data, while the second data structure 108 can include domain-specific data (which may be limited in access as compared with the data of the first data structure 108). The system 100 may use candidate internal donors 112 from the same or different data structures 108 to configure, train, update, and/or perform runtime operations on candidate internal donors 112.

In some instances, a given candidate internal donor chemical structure 112 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 112 include SMILES representations, the system 100 may include all SMILES enumerations of the desired chemical species as input.

In addition to the internal donor chemical structure 112, the data structure 108 may include as input to the machine learning model 104 one or more additional parameters. These additional parameters may include one or more polymerization parameters, one or more polyolefin properties, a confidence score, or a combination thereof. The confidence score may indicate a likelihood that the predicted internal donor percentage for the corresponding candidate internal donor 112 is accurate.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical properties, a three-dimensional (3D) descriptor, or a combination thereof. The additional parameters may include one or more of (i) a set of calculated physical properties, and (ii) a set of calculated three-dimensional (3D) descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor.

A 2D descriptor is a descriptor calculated from two-dimensional information about atoms constituting a compound of an external electron donor, how atoms are connected, and the like. The 3D descriptor is a descriptor calculated based on three-dimensional structural information such as coordinates of atoms constituting the compound of the external electron donor, interatomic distances, and bond angles. The above structural parameters can be calculated from the molecular structure of the external electron donor using programs such as Mordred and RDKit.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF (characterizes complexity of a molecule by taking the ratio of the number of heavy atoms in the Murcko framework of the molecule to the total number of heavy atoms in the molecule, useful for similarity analysis and property prediction), fragment complexity, hydrogen bond acceptor count, hydrogen bond donor count, hybridization ratio descriptor, khs.aaaCKierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12 MDE, MDEC-13 MDE, MDEC-14 MDE, MDEC-22 MDE, MDEC-23 MDE, MDEC-24 MDE, MDEC-33 MDE, MDEC-34 MDE, MDEC-44 MDE, MDEN-11 MDE, MDEN-12 MDE, MDEN-13 MDE, MDEN-22 MDE, MDEN-23 MDE, MDEN-33 MDE, MDEO-11 MDE, MDEO-12 MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

The output 114 from the machine learning model 104 may include a predicted internal donor percentage for the respective candidate internal donor 112. The predicted internal donor percentage may be a value or a range of values representing the relative amount of the candidate internal donor 112 that is added during preparation of the procatalyst. For example, the predicted internal donor percentage value 114 may be about 0.01 wt. % to about 90 wt. % (e.g., about 0.1 wt. % to about 50 wt. %, about 0.5 wt. % to about 25 wt. %, about 5 wt. % to about 22 wt. %, about 8 wt. % to about 22 wt. %, about 8 wt. % to about 15 wt. %, about 10 wt. % to about 16 wt. %, or any value or subrange therebetween). The output 114 may include one or more plots based on the output of the machine learning model 104.

In addition to or instead of predicted internal donor percentage, the output 114 may include one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The system may include one or more training examples 120. The training examples 120 may be maintained by one or more entities, which may be entities that maintain the system 100 and/or may be separate from entities that maintain the system 100. For example, the training examples 120 may include publicly available data. The system 100 may use training examples 120 from the same or different data sets to configure, train, update, and/or perform runtime operations on candidate internal donors 112.

The training example 120 may include a chemical structure 122 and a corresponding internal donor percentage value 124. For example, the chemical structure 122 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 122 may be represented by multiple different data structures (e.g., different strings or enumerations). For example, multiple strings may be used to represent a single chemical species in order to improve the representation of that chemical species and describe the chemical species with all possible angles. Where the chemical structure 122 may include multiple strings that represent the same chemical species, the chemical structure may include some or all of the multiple strings that represent the same chemical species. For example, where the chemical structure 122 includes SMILES representations, the system 100 may include all SMILES enumerations of the desired chemical species as input.

The corresponding internal donor percentage value 124 may be a value or a range of values representing the relative amount of the internal donor represented by the chemical structure 122 that is added during preparation of the procatalyst.

In addition to the corresponding internal donor percentage value 124, the training example 120 may include as input to the machine learning model 104 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical properties, a two-dimensional (2D) descriptor, a three-dimensional (3D) descriptor, or a combination of any two or more thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12 MDE, MDEC-13 MDE, MDEC-14 MDE, MDEC-22 MDE, MDEC-23 MDE, MDEC-24 MDE, MDEC-33 MDE, MDEC-34 MDE, MDEC-44 MDE, MDEN-11 MDE, MDEN-12 MDE, MDEN-13 MDE, MDEN-22 MDE, MDEN-23 MDE, MDEN-33 MDE, MDEO-11 MDE, MDEO-12 MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

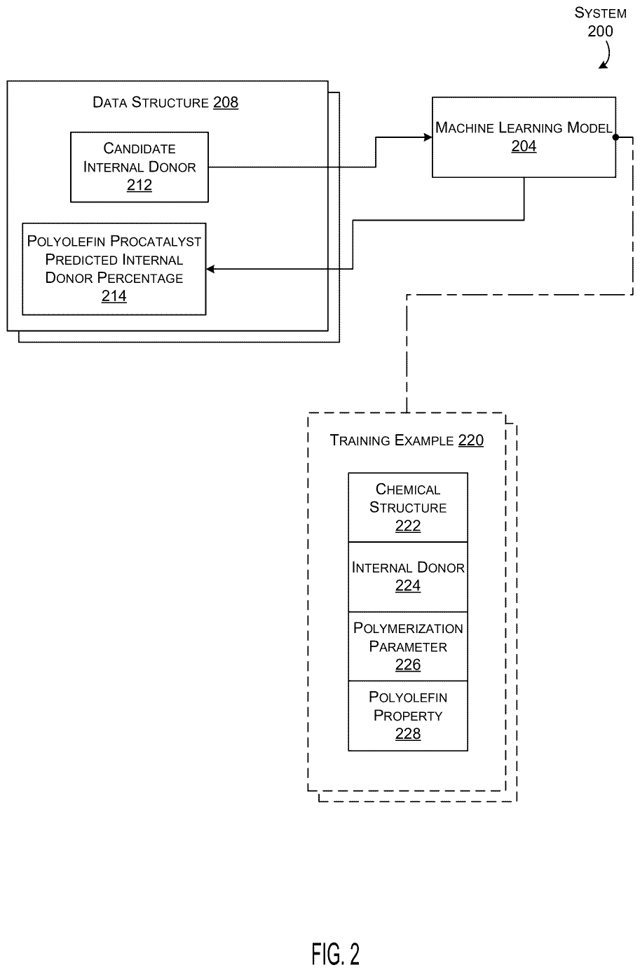

Referring to FIG. 2, illustrated is a block diagram of an example system 200 for predicting polyolefin procatalyst internal donor percentage using a machine learning model updated using a plurality of training examples, each training example including chemical structure and respective internal donor percentage.

Operations described in connection with FIG. 2 may be performed via one or more computing devices. For example, the computer system 900 of FIG. 9 may implement some or all of the operations described herein, through the execution of computer-readable instructions.

The system 200 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 200 may include one or more computing devices or servers that can perform various functions as described herein.

The system 200 may execute one or more machine learning models 204 in response to corresponding request(s) from an operator of the system 200 or from an external computing system. Outputs from the system 200 may be displayed on a display device of a computing device executing the system 200. For example, the computer system 900 of FIG. 9 may output predicted percent donor values and may display these values on display 914.

The system 200 may include one or more data structures 208, which may be stored in a storage device (e.g., storage device 910 of computer system 900 in FIG. 9), such as a solid-state device, magnetic disk, or optical disk for persistently storing information and instructions. The data structures 208 may be received from the storage device by a processor (e.g., processor 904 of computer system 900 in FIG. 9) via a bus (e.g., bus 902 of computer system 900 in FIG. 9).

The system 200 may include one or more machine learning models 204, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 200 may include one or more training examples 220, which may be stored in a storage device (e.g., storage device 910 of computer system 900 in FIG. 9), such as a solid-state device, magnetic disk, or optical disk for persistently storing information and instructions. The storage device in which the training examples 220 are stored may be the same or different than the storage device for storing the data structures 208. The training examples 220 may be received from the storage device by the processor (e.g., processor 904 of computer system 900 in FIG. 9) via the bus (e.g., bus 902 of computer system 900 in FIG. 9).

In some implementations, components of the system 200 may be implemented using multiple computing systems in communication with one other, for example, via a network. In some implementations, the system 200 may be implemented at least in part of a distributed computing system For example, the system 200 may be or may include an application server or webserver, which may include software modules to access or transmit data structures 208 stored by the memory in the system 200. In some implementations, one or more client devices of the system 200 may transmit data structures 208 to the distributed computing system in response to user input by an operator of the client devices. Such requests may include requests to initiating training and/or inference operations based on data submitted to the computing system maintaining the machine-learning model 204.

The system 200 may include a network to facilitate communication between remote components of the system 200 and/or between the system 200 and a remote computing system. The network may include any number of hardwired or wireless connections. Any or all of the computing devices described herein (e.g., the system 200) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 200) may also communicate wirelessly with other computing devices via a proxy device (e.g., a router, network switch, or gateway).

The system 200 may identify a data structure 208 comprising one or more chemical structures as candidate internal donors 212 for polyolefin procatalysts. The system may provide the data structure 208 as input to a model 204 to generate a predicted internal donor percentage for each resulting polyolefin procatalyst created using each chemical structure of the one or more chemical structures in the data structure 208. The system 200 may provide, for presentation, an output 214 including the predicted internal donor percentage for each of the one or more chemical structures for the internal donor.

The model 204 may be effective for operating on or generating data representative of chemical structures. For example, the candidate internal donor 212 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 200 includes one or more models 204. The models 204 may be trained using one or more training examples. The model 204 may include machine learning models or other models that can generate target outputs based on various types of inputs.

The system 200 may train, update, and/or configure one or more models 204. The model 204 may include machine learning models or other models that may generate target outputs based on various types of inputs. The system 200 may train, update, and/or configure the model 204 by modifying or updating one or more parameters responsive to evaluating one or more training examples 220 in view of expected or ground truth outputs.

The system 200 may operate on one or more candidate internal donors 212 (e.g., inference or runtime data; training data), which may be retrieved from one or more data structures 208. The data structure may include various databases, molecule editors, sensor data streams, user input sources, or a combination of any two or more thereof from which the system 200 can retrieve candidate internal donor 212 to process.

The data structure 208 may be maintained by one or more entities, which may be entities that maintain the system 200 and/or may be separate from entities that maintain the system 200. In some implementations, the system 200 uses data from different data sets, such as by using candidate internal donors 212 from a first data structure 208 to perform at least a first configuring (e.g., updating or training) of the model 204, and uses training data elements from a second data structure 208 to perform at least a second configuring of the models 204. For example, the first data structure 208 may include publicly available data, while the second data structure 208 can include domain-specific data (which may be limited in access as compared with the data of the first data structure 208). The system 200 may use candidate internal donors 212 from the same or different data structures 208 to configure, train, update, and/or perform runtime operations on candidate internal donors 212.

In some instances, a given candidate internal donor chemical structure 212 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 212 include SMILES representations, the system 200 may include all SMILES enumerations of the desired chemical species as input.

In addition to the internal donor chemical structure 212, the data structure 208 may include as input to the machine learning model 204 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical property, a two-dimensional (2D) descriptor, a three-dimensional (3D) descriptor, or a combination of any two or more thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12 MDE, MDEC-13 MDE, MDEC-14 MDE, MDEC-22 MDE, MDEC-23 MDE, MDEC-24 MDE, MDEC-33 MDE, MDEC-34 MDE, MDEC-44 MDE, MDEN-11 MDE, MDEN-12 MDE, MDEN-13 MDE, MDEN-22 MDE, MDEN-23 MDE, MDEN-33 MDE, MDEO-11 MDE, MDEO-12 MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

The output 214 from the machine learning model 204 may include a predicted internal donor percentage for the respective candidate internal donor 212. The predicted internal donor percentage may be a value or a range of values representing the relative amount of the candidate internal donor 212 that is added during preparation of the procatalyst.

In addition to or instead of predicted internal donor percentage, the output 214 may include one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The training example 220 may include a chemical structure 222, a corresponding internal donor percentage value 224, a polymerization parameter 226, and a polyolefin property 228. For example, the chemical structure 222 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 222 may be represented by multiple different data structures (e.g., different strings or enumerations). For example, multiple strings may be used to represent a single chemical species in order to improve the representation of that chemical species and describe the chemical species with all possible angles. Where the chemical structure 222 may include multiple strings that represent the same chemical species, the chemical structure may include some or all of the multiple strings that represent the same chemical species. For example, where the chemical structure 222 includes SMILES representations, the system 200 may include all SMILES enumerations of the desired chemical species as input.

The corresponding internal donor percentage value 224 may be a value or a range of values representing the relative amount of the internal donor represented by the chemical structure 222 that is added during preparation of the procatalyst.

The training example 220 may include one or more corresponding polymerization parameters 226. The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value

The training example 220 may include one or more corresponding polyolefin properties 228. The one or more polyolefin properties 228 comprise one or more of a xylene solubility (XS) value, a polydispersity index (PDI) value, or a melt flow response (MFR) value.

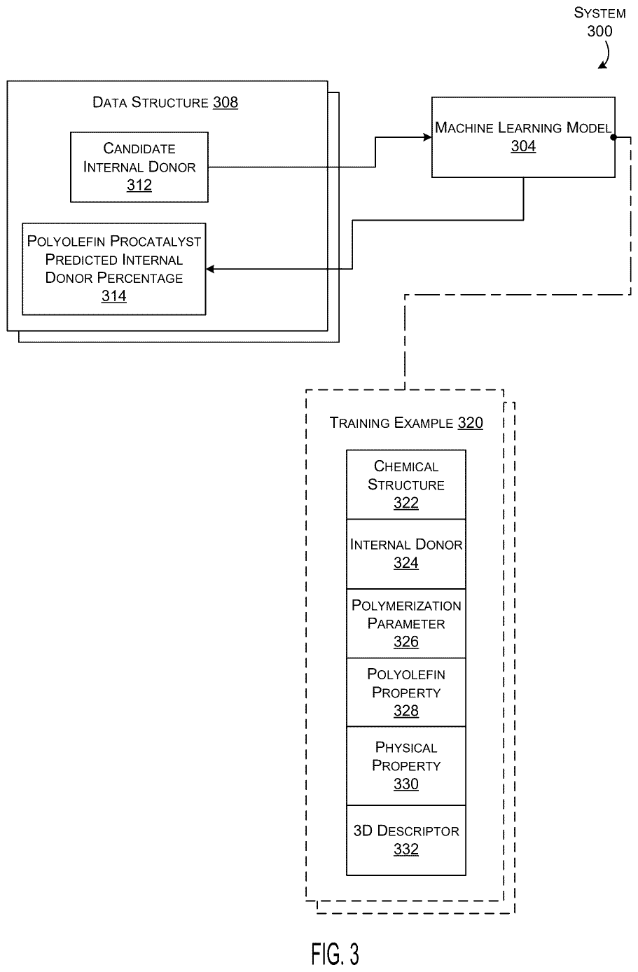

Referring to FIG. 3, illustrated is a block diagram of an example system 300 for predicting polyolefin procatalyst internal donor percentage using a machine learning model updated using a plurality of training examples, each training example including chemical structure and respective internal donor percentage.

Operations described in connection with FIG. 3 may be performed via one or more computing devices. For example, the computer system 900 of FIG. 9 may implement some or all of the operations described herein, through the execution of computer-readable instructions.

The system 300 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 300 may include one or more computing devices or servers that can perform various functions as described herein.

The system 300 may execute one or more machine learning models 304 in response to corresponding request(s) from an operator of the system 300 or from an external computing system. Outputs from the system 300 may be displayed on a display device of a computing device executing the system 300. For example, the computer system 900 of FIG. 9 may output predicted percent donor values and may display these values on display 914.

The system 300 may include one or more data structures 308, which may be stored in a storage device (e.g., storage device 910 of computer system 900 in FIG. 9), such as a solid-state device, magnetic disk, or optical disk for persistently storing information and instructions. The data structures 308 may be received from the storage device by a processor (e.g., processor 904 of computer system 900 in FIG. 9) via a bus (e.g., bus 902 of computer system 900 in FIG. 9).

The system 300 may include one or more machine learning models 304, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 300 may include one or more training examples 320, which may be stored in a storage device (e.g., storage device 910 of computer system 900 in FIG. 9), such as a solid-state device, magnetic disk, or optical disk for persistently storing information and instructions. The storage device in which the training examples 320 are stored may be the same or different than the storage device for storing the data structures 308. The training examples 320 may be received from the storage device by the processor (e.g., processor 904 of computer system 900 in FIG. 9) via the bus (e.g., bus 902 of computer system 900 in FIG. 9).

In some implementations, components of the system 300 may be implemented using multiple computing systems in communication with one other, for example, via a network. In some implementations, the system 300 may be implemented at least in part of a distributed computing system. For example, the system 300 may be or may include an application server or webserver, which may include software modules to access or transmit data structures 308 stored by the memory in the system 300. In some implementations, one or more client devices of the system 300 may transmit data structures 308 to the distributed computing system in response to user input by an operator of the client devices. Such requests may include requests to initiating training and/or inference operations based on data submitted to the computing system maintaining the machine-learning model 304.

The system 300 may include a network to facilitate communication between remote components of the system 300 and/or between the system 300 and a remote computing system. The network may include any number of hardwired or wireless connections. Any or all of the computing devices described herein (e.g., the system 300) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 300) may also communicate wirelessly with other computing devices via a proxy device (e.g., a router, network switch, or gateway).

The system 300 may identify a data structure 308 comprising one or more chemical structures as candidate internal donors 312 for polyolefin procatalysts. The system may provide the data structure 308 as input to a model 304 to generate a predicted internal donor percentage for each resulting polyolefin procatalyst created using each chemical structure of the one or more chemical structures in the data structure 308. The system 300 may provide, for presentation, an output 314 including the predicted internal donor percentage for each of the one or more chemical structures for the internal donor.

The model 304 may be effective for operating on or generating data representative of chemical structures. For example, the candidate internal donor 312 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 300 includes one or more models 304. The models 304 may be trained using one or more training examples. The model 304 may include machine learning models or other models that can generate target outputs based on various types of inputs.

The system 300 may train, update, and/or configure one or more models 304. The model 304 may include machine learning models or other models that may generate target outputs based on various types of inputs. The system 300 may train, update, and/or configure the model 304 by modifying or updating one or more parameters responsive to evaluating one or more training examples 320 in view of expected or ground truth outputs.

The system 300 may operate on one or more candidate internal donors 312 (e.g., inference or runtime data; training data), which may be retrieved from one or more data structures 308. The data structure may include various databases, molecule editors, sensor data streams, user input sources, or a combination of any two or more thereof from which the system 300 can retrieve candidate internal donor 312 to process.

The data structure 308 may be maintained by one or more entities, which may be entities that maintain the system 300 and/or may be separate from entities that maintain the system 300. In some implementations, the system 300 uses data from different data sets, such as by using candidate internal donors 312 from a first data structure 308 to perform at least a first configuring (e.g., updating or training) of the model 304, and uses training data elements from a second data structure 308 to perform at least a second configuring of the models 304. For example, the first data structure 308 may include publicly available data, while the second data structure 308 can include domain-specific data (which may be limited in access as compared with the data of the first data structure 308). The system 300 may use candidate internal donors 312 from the same or different data structures 308 to configure, train, update, and/or perform runtime operations on candidate internal donors 312.

In some instances, a given candidate internal donor chemical structure 312 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 312 include SMILES representations, the system 300 may include all SMILES enumerations of the desired chemical species as input.

In addition to the internal donor chemical structure 312, the data structure 308 may include as input to the machine learning model 304 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical properties, a two-dimensional (2D) descriptor, a three-dimensional (3D) descriptor, or a combination of any two or more thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12 MDE, MDEC-13 MDE, MDEC-14 MDE, MDEC-22 MDE, MDEC-23 MDE, MDEC-24 MDE, MDEC-33 MDE, MDEC-34 MDE, MDEC-44 MDE, MDEN-11 MDE, MDEN-12 MDE, MDEN-13 MDE, MDEN-22 MDE, MDEN-23 MDE, MDEN-33 MDE, MDEO-11 MDE, MDEO-12 MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

The output 314 from the machine learning model 304 may include a predicted internal donor percentage for the respective candidate internal donor 312. The predicted internal donor percentage may be a value or a range of values representing the relative amount of the candidate internal donor 312 that is added during preparation of the procatalyst.

In addition to or instead of predicted internal donor percentage, the output 314 may include one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The training example 320 may include a chemical structure 322, a corresponding internal donor percentage value 324, a polymerization parameter 326, a polyolefin property 328, a physical property 330, and a 2D and/or 3D descriptor 332. For example, the chemical structure 322 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 322 may be represented by multiple different data structures (e.g., different strings or enumerations). For example, multiple strings may be used to represent a single chemical species in order to improve the representation of that chemical species and describe the chemical species with all possible angles. Where the chemical structure 322 may include multiple strings that represent the same chemical species, the chemical structure may include some or all of the multiple strings that represent the same chemical species. For example, where the chemical structure 322 includes SMILES representations, the system 300 may include all SMILES enumerations of the desired chemical species as input.

The corresponding internal donor percentage value 324 may be a value or a range of values representing the relative amount of the internal donor represented by the chemical structure 322 that is added during preparation of the procatalyst.

The training example 320 may include one or more corresponding polymerization parameters 326. The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value

The training example 320 may include one or more corresponding polyolefin properties 328. The one or more polyolefin properties 328 comprise one or more of a xylene solubility (XS) value, a polydispersity index (PDI) value, or a melt flow response (MFR) value.

The training example 320 may include one or more corresponding physical properties 330. The training example 320 may include one or more parameters from a set of calculated physical properties. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The training example 320 may include one or more corresponding 2D and/or 3D descriptors 332 relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The training example 320 may include one or more corresponding 3D descriptors 332. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12 MDE, MDEC-13 MDE, MDEC-14 MDE, MDEC-22 MDE, MDEC-23 MDE, MDEC-24 MDE, MDEC-33 MDE, MDEC-34 MDE, MDEC-44 MDE, MDEN-11 MDE, MDEN-12 MDE, MDEN-13 MDE, MDEN-22 MDE, MDEN-23 MDE, MDEN-33 MDE, MDEO-11 MDE, MDEO-12 MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

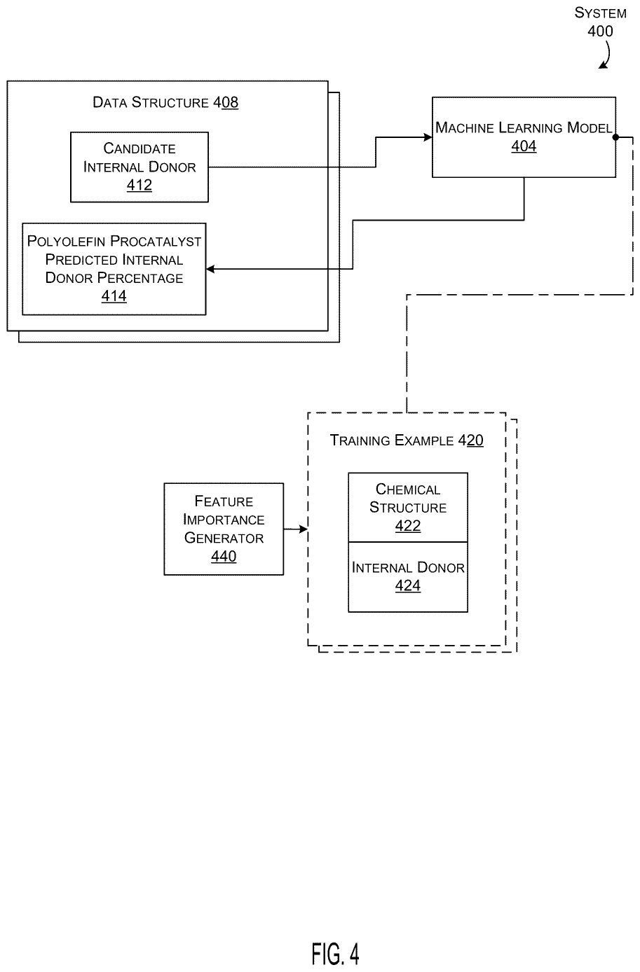

Referring to FIG. 4, illustrated is a block diagram of an example system 400 for predicting polyolefin procatalyst internal donor percentage using a machine learning model updated using a plurality of training examples, each training example including chemical structure and respective internal donor percentage.

Operations described in connection with FIG. 4 may be performed via one or more computing devices. For example, the computer system 900 of FIG. 9 may implement some or all of the operations described herein, through the execution of computer-readable instructions.

The system 400 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 400 may include one or more computing devices or servers that can perform various functions as described herein.

The system 400 may execute one or more machine learning models 404 in response to corresponding request(s) from an operator of the system 400 or from an external computing system. Outputs from the system 400 may be displayed on a display device of a computing device executing the system 400. For example, the computer system 900 of FIG. 9 may output predicted percent donor values and may display these values on display 914.

The system 400 may include one or more data structures 408, which may be stored in a storage device (e.g., storage device 910 of computer system 900 in FIG. 9), such as a solid-state device, magnetic disk, or optical disk for persistently storing information and instructions. The data structures 408 may be received from the storage device by a processor (e.g., processor 904 of computer system 900 in FIG. 9) via a bus (e.g., bus 902 of computer system 900 in FIG. 9).

The system 400 may include one or more machine learning models 404, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 400 may include one or more training examples 420, which may be stored in a storage device (e.g., storage device 910 of computer system 900 in FIG. 9), such as a solid-state device, magnetic disk, or optical disk for persistently storing information and instructions. The storage device in which the training examples 420 are stored may be the same or different than the storage device for storing the data structures 408. The training examples 420 may be received from the storage device by the processor (e.g., processor 904 of computer system 900 in FIG. 9) via the bus (e.g., bus 902 of computer system 900 in FIG. 9).

In some implementations, components of the system 400 may be implemented using multiple computing systems in communication with one other, for example, via a network. In some implementations, the system 400 may be implemented at least in part of a distributed computing system For example, the system 400 may be or may include an application server or webserver, which may include software modules to access or transmit data structures 408 stored by the memory in the system 400. In some implementations, one or more client devices of the system 400 may transmit data structures 408 to the distributed computing system in response to user input by an operator of the client devices. Such requests may include requests to initiating training and/or inference operations based on data submitted to the computing system maintaining the machine-learning model 404.

The system 400 may include a network to facilitate communication between remote components of the system 400 and/or between the system 400 and a remote computing system. The network may include any number of hardwired or wireless connections. Any or all of the computing devices described herein (e.g., the system 400) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 400) may also communicate wirelessly with other computing devices via a proxy device (e.g., a router, network switch, or gateway).

The system 400 may identify a data structure 408 comprising one or more chemical structures as candidate internal donors 412 for polyolefin procatalysts. The system may provide the data structure 408 as input to a model 404 to generate a predicted internal donor percentage for each resulting polyolefin procatalyst created using each chemical structure of the one or more chemical structures in the data structure 408. The system 400 may provide, for presentation, an output 414 including the predicted internal donor percentage for each of the one or more chemical structures for the internal donor.

The model 404 may be effective for operating on or generating data representative of chemical structures. For example, the candidate internal donor 412 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 400 includes one or more models 404. The models 404 may be trained using one or more training examples. The model 404 may include machine learning models or other models that can generate target outputs based on various types of inputs.

The system 400 may train, update, and/or configure one or more models 404. The model 404 may include machine learning models or other models that may generate target outputs based on various types of inputs. The system 400 may train, update, and/or configure the model 404 by modifying or updating one or more parameters responsive to evaluating one or more training examples 420 in view of expected or ground truth outputs.

The system 400 may operate on one or more candidate internal donors 412 (e.g., inference or runtime data; training data), which may be retrieved from one or more data structures 408. The data structure may include various databases, molecule editors, sensor data streams, user input sources, or a combination of any two or more thereof from which the system 400 can retrieve candidate internal donor 412 to process.

The data structure 408 may be maintained by one or more entities, which may be entities that maintain the system 400 and/or may be separate from entities that maintain the system 400. In some implementations, the system 400 uses data from different data sets, such as by using candidate internal donors 412 from a first data structure 408 to perform at least a first configuring (e.g., updating or training) of the model 404, and uses training data elements from a second data structure 408 to perform at least a second configuring of the models 404. For example, the first data structure 408 may include publicly available data, while the second data structure 408 can include domain-specific data (which may be limited in access as compared with the data of the first data structure 408). The system 400 may use candidate internal donors 412 from the same or different data structures 408 to configure, train, update, and/or perform runtime operations on candidate internal donors 412.

In some instances, a given candidate internal donor chemical structure 412 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 412 include SMILES representations, the system 400 may include all SMILES enumerations of the desired chemical species as input.

In addition to the internal donor chemical structure 412, the data structure 408 may include as input to the machine learning model 404 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical property, a two-dimensional (2D) descriptor, a three-dimensional (3D) descriptor, or a combination of any two or more thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12MDE, MDEC-13 MDE, MDEC-14MDE, MDEC-22 MDE, MDEC-23 MDE, MDEC-24 MDE, MDEC-33 MDE, MDEC-34 MDE, MDEC-44 MDE, MDEN-11 MDE, MDEN-12MDE, MDEN-13 MDE, MDEN-22 MDE, MDEN-23 MDE, MDEN-33 MDE, MDEO-11 MDE, MDEO-12MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

The output 414 from the machine learning model 404 may include a predicted internal donor percentage for the respective candidate internal donor 412. The predicted internal donor percentage may be a value or a range of values representing the relative amount of the candidate internal donor 412 that is added during preparation of the procatalyst.

In addition to or instead of predicted internal donor percentage, the output 414 may include one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The training example 420 may include a chemical structure 422 and a corresponding internal donor percentage value 424. For example, the chemical structure 422 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 422 may be represented by multiple different data structures (e.g., different strings or enumerations). For example, multiple strings may be used to represent a single chemical species in order to improve the representation of that chemical species and describe the chemical species with all possible angles. Where the chemical structure 422 may include multiple strings that represent the same chemical species, the chemical structure may include some or all of the multiple strings that represent the same chemical species. For example, where the chemical structure 422 includes SMILES representations, the system 400 may include all SMILES enumerations of the desired chemical species as input.

The corresponding internal donor percentage value 424 may be a value or a range of values representing the relative amount of the internal donor represented by the chemical structure 422 that is added during preparation of the procatalyst.

In addition to the corresponding internal donor percentage value 424, the training example 420 may include as input to the machine learning model 404 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical properties, a two-dimensional (2D) descriptor, a three-dimensional (3D) descriptor, or a combination thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12MDE, MDEC-13 MDE, MDEC-14MDE, MDEC-22 MDE, MDEC-23 MDE, MDEC-24 MDE, MDEC-33 MDE, MDEC-34 MDE, MDEC-44 MDE, MDEN-11 MDE, MDEN-12MDE, MDEN-13 MDE, MDEN-22 MDE, MDEN-23 MDE, MDEN-33 MDE, MDEO-11 MDE, MDEO-12MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

The system 400 may include a feature importance generator 440. The feature importance generator 440 may generate a feature importance for one or more values of a dataset. The feature importance generator 440 may generate the plurality of training examples 420 based on the feature importance. The feature importance generator 440 may rank one or more values of a dataset according to feature importance. Feature importance of one or more values of a dataset may be evaluated for the importance of one or more parameters (e.g., internal donor percentage, polymerization parameter, polyolefin property, physical property, 2D descriptor, 3D descriptor, or a combination of any two or more thereof).

The feature importance generator 440 may be included in the one or more models 404 or may be another model. The model may be trained using one or more training examples. The model may include machine learning models or other models that can generate target outputs based on various types of inputs. The feature importance generator 440 may be trained on chemistry principals and may generate a feature importance based on alignment with chemistry principals. The feature importance generator 440 may generate a feature importance for one or more parameters (e.g., internal donor percentage, polymerization parameter, polyolefin property, physical property, 2D descriptor, 3D descriptor, or a combination of any two or more thereof) based on another parameter. For example, the feature importance generator 440 may generate a feature importance for one or more parameters (e.g., polymerization parameter, polyolefin property, physical property, 2D descriptor, 3D descriptor, or a combination of any two or more thereof) based one internal donor percentage.

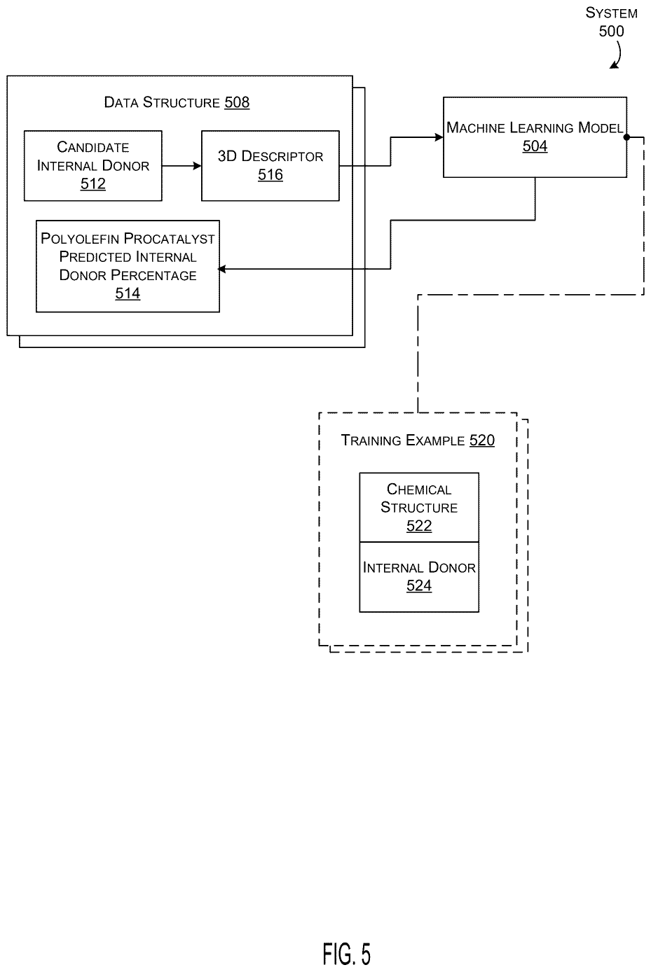

Referring to FIG. 5, illustrated is a block diagram of an example system 500 for predicting polyolefin procatalyst internal donor percentage using a machine learning model updated using a plurality of training examples, each training example including chemical structure and respective internal donor percentage.

Operations described in connection with FIG. 5 may be performed via one or more computing devices. For example, the computer system 900 of FIG. 9 may implement some or all of the operations described herein, through the execution of computer-readable instructions.

The system 500 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 500 may include one or more computing devices or servers that can perform various functions as described herein.

The system 500 may execute one or more machine learning models 504 in response to corresponding request(s) from an operator of the system 500 or from an external computing system. Outputs from the system 500 may be displayed on a display device of a computing device executing the system 500. For example, the computer system 900 of FIG. 9 may output predicted percent donor values and may display these values on display 914.

The system 500 may include one or more data structures 508, which may be stored in a storage device (e.g., storage device 910 of computer system 900 in FIG. 9), such as a solid-state device, magnetic disk, or optical disk for persistently storing information and instructions. The data structures 508 may be received from the storage device by a processor (e.g., processor 904 of computer system 900 in FIG. 9) via a bus (e.g., bus 902 of computer system 900 in FIG. 9).

The system 500 may include one or more machine learning models 504, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 500 may include one or more training examples 520, which may be stored in a storage device (e.g., storage device 910 of computer system 900 in FIG. 9), such as a solid-state device, magnetic disk, or optical disk for persistently storing information and instructions. The storage device in which the training examples 520 are stored may be the same or different than the storage device for storing the data structures 508. The training examples 520 may be received from the storage device by the processor (e.g., processor 904 of computer system 900 in FIG. 9) via the bus (e.g., bus 902 of computer system 900 in FIG. 9).

In some implementations, components of the system 500 may be implemented using multiple computing systems in communication with one other, for example, via a network. In some implementations, the system 500 may be implemented at least in part of a distributed computing system For example, the system 500 may be or may include an application server or webserver, which may include software modules to access or transmit data structures 508 stored by the memory in the system 500. In some implementations, one or more client devices of the system 500 may transmit data structures 508 to the distributed computing system in response to user input by an operator of the client devices. Such requests may include requests to initiating training and/or inference operations based on data submitted to the computing system maintaining the machine-learning model 504.

The system 500 may include a network to facilitate communication between remote components of the system 500 and/or between the system 500 and a remote computing system. The network may include any number of hardwired or wireless connections. Any or all of the computing devices described herein (e.g., the system 500) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 500) may also communicate wirelessly with other computing devices via a proxy device (e.g., a router, network switch, or gateway).

The system 500 may identify a data structure 508 comprising one or more chemical structures as candidate internal donors 512 for polyolefin procatalysts. The system may provide the data structure 508 as input to a model 504 to generate a predicted internal donor percentage for each resulting polyolefin procatalyst created using each chemical structure of the one or more chemical structures in the data structure 508. The system 500 may provide, for presentation, an output 514 including the predicted internal donor percentage for each of the one or more chemical structures for the internal donor.

The model 504 may be effective for operating on or generating data representative of chemical structures. For example, the candidate internal donor 512 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 500 includes one or more models 504. The models 504 may be trained using one or more training examples. The model 504 may include machine learning models or other models that can generate target outputs based on various types of inputs.

The system 500 may train, update, and/or configure one or more models 504. The model 504 may include machine learning models or other models that may generate target outputs based on various types of inputs. The system 500 may train, update, and/or configure the model 504 by modifying or updating one or more parameters responsive to evaluating one or more training examples 520 in view of expected or ground truth outputs.

The system 500 may operate on one or more candidate internal donors 512 (e.g., inference or runtime data; training data), which may be retrieved from one or more data structures 508. The data structure may include various databases, molecule editors, sensor data streams, user input sources, or a combination of any two or more thereof from which the system 500 can retrieve candidate internal donor 512 to process. The system 500 may operate on one or more 2D and/or 3D descriptors 516 associated with corresponding candidate internal donors 512.

The data structure 508 may be maintained by one or more entities, which may be entities that maintain the system 500 and/or may be separate from entities that maintain the system 500. In some implementations, the system 500 uses data from different data sets, such as by using candidate internal donors 512 and/or 2D and/or 3D descriptor 516 from a first data structure 508 to perform at least a first configuring (e.g., updating or training) of the model 504, and uses training data elements from a second data structure 508 to perform at least a second configuring of the models 504. For example, the first data structure 508 may include publicly available data, while the second data structure 508 can include domain-specific data (which may be limited in access as compared with the data of the first data structure 508). The system 500 may use candidate internal donors 512 and/or 2D and/or 3D descriptor 516 from the same or different data structures 508 to configure, train, update, and/or perform runtime operations on candidate internal donors 512.

In some instances, a given candidate internal donor chemical structure 512 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 512 include SMILES representations, the system 500 may include all SMILES enumerations of the desired chemical species as input.

The one or more descriptors 516 may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The one or more 3D descriptors 516 may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12MDE, MDEC-13 MDE, MDEC-14MDE, MDEC-22 MDE, MDEC-23 MDE, MDEC-24 MDE, MDEC-33 MDE, MDEC-34 MDE, MDEC-44 MDE, MDEN-11 MDE, MDEN-12MDE, MDEN-13 MDE, MDEN-22 MDE, MDEN-23 MDE, MDEN-33 MDE, MDEO-11 MDE, MDEO-12MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

In addition to the internal donor chemical structure 512, the data structure 508 may include as input to the machine learning model 504 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include one or more physical properties. The additional parameters may include a set of calculated physical properties. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The output 514 from the machine learning model 504 may include a predicted internal donor percentage for the respective candidate internal donor 512. The predicted internal donor percentage may be a value or a range of values representing the relative amount of the candidate internal donor 512 that is added during preparation of the procatalyst.

In addition to or instead of predicted internal donor percentage, the output 514 may include one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The training example 520 may include a chemical structure 522 and a corresponding internal donor percentage value 524. For example, the chemical structure 522 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 522 may be represented by multiple different data structures (e.g., different strings or enumerations). For example, multiple strings may be used to represent a single chemical species in order to improve the representation of that chemical species and describe the chemical species with all possible angles. Where the chemical structure 522 may include multiple strings that represent the same chemical species, the chemical structure may include some or all of the multiple strings that represent the same chemical species. For example, where the chemical structure 522 includes SMILES representations, the system 500 may include all SMILES enumerations of the desired chemical species as input.

The corresponding internal donor percentage value 524 may be a value or a range of values representing the relative amount of the internal donor represented by the chemical structure 522 that is added during preparation of the procatalyst.

In addition to the corresponding internal donor percentage value 524, the training example 520 may include as input to the machine learning model 504 one or more additional parameters. These additional parameters may include one or more polymerization parameters, one or more polyolefin properties, or a combination thereof.

The one or more polymerization parameters may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The additional parameters may include a physical properties, a two-dimensional (2D) descriptor, a three-dimensional (3D) descriptor, or a combination of any two or more thereof. The additional parameters may include one or more of (i) a set of calculated physical properties, and (ii) a set of calculated 2D and/or 3D descriptors. The set of calculated physical properties may include one or more values derived from density functional theory (DFT) calculations. For example, the values derived from DFT calculations may include one or more of an electronegativity value, a hardness value, a dipole moment, a highest occupied molecular orbital (HOMO), and a lowest unoccupied molecular orbital (LUMO).

The additional parameters may include one or more 2D and/or 3D structural descriptors relating to the molecular structure of the external electron donor, as described herein.

The 2D descriptors include, for example, the number of atoms constituting the external electron donor, the number of bonds, functional group characteristics, Kier & Hall Connectivity Index, Kappa Shape Index, BCUT/GCUT descriptors, estimated volume/surface area (volume, surface area, TPSA, VSA), physicochemical properties (molecular weight, logP, logS, molar refraction, electric charge, etc.), drug-like, lead-like, reactivity, mutagenicity, synthesis possibility, description based on the extended Huckel method Child, ABCIndex, AcidBase, AdjacencyMatrix, Aromatic, AtomCount, Autocorrelation, BalabanJ, BaryszMatrix, BertzCT, BondCount, CarbonTypes, Chi, Constitutional, DetourMatrix, DistanceMatrix, EccentricConnectivityIndex, Estate, ExtendedTopochemicalAtom, FragmentComplexity, Framework, HydrogenBond, InformationContent, Lipinski, McGowanVolume, MoeType, MolecularDistanceEdge, MolecularId, PathCount, Polarizability, RingCount, RotatableBond, TopologicalCharge, TopologicalIndex, TopoPSA, VdwVolumeABC, Verte It may include one or more selected from xAdjacencyInformation, WalkCount, WienerIndex, ZagrebIndex, and the like.

The 3D descriptors are, for example, external electron donor volume, surface area, shape descriptor, dipole moment, radius of gyration, sphericity, potential energy, strain energy, interaction energy, VolSur descriptor, MOPAC descriptor, CPSA, GeometricIndex, GravitationalIndex, MomentOfInertia, MoRSE, and the like.

The additional parameters may include one or more 3D descriptors. The 3D descriptors may include one or more of acid group count, ALOGP, ALOGP2, AMR ALOGP, aromatic atom count, aromatic bond count, atom count, atomic polarizability, Broto-Moreau autocorrelation of a topological structure-lag1/weighted by atomic masses (aTSm1), Broto-Moreau autocorrelation of a topological structure-lag2/weighted by atomic masses (aTSm2), Broto-Moreau autocorrelation of a topological structure-lag3/weighted by atomic masses (aTSm3), Broto-Moreau autocorrelation of a topological structure-lag4/weighted by atomic masses (aTSm4), Broto-Moreau autocorrelation of a topological structure-lag5/weighted by atomic masses (aTSm5), Broto-Moreau autocorrelation of a topological structure-lag1/weighted by atomic polarizabilities (ATSp1), Broto-Moreau autocorrelation of a topological structure-lag2/weighted by atomic polarizabilities (ATSp2), Broto-Moreau autocorrelation of a topological structure-lag3/weighted by atomic polarizabilities (ATSp3), Broto-Moreau autocorrelation of a topological structure-lag4/weighted by atomic polarizabilities (ATSp4), Broto-Moreau autocorrelation of a topological structure-lag5/weighted by atomic polarizabilities (ATSp5), basic group count, bond count, bpol, C1SP1, C1SP2, C1SP3, C2SP1, C2SP2, C3SP2, C3SP3, C4SP3, eccentric connectivity index, FMF, fragment complexity, hydrogen bond acceptor count, hydrogen bond donor count, hybridization ratio descriptor, khs.aaaC KierHallSmarts, khs.aaCH KierHallSmarts, khs.aaN KierHallSmarts, khs.aaNH KierHallSmarts, khs.aaO KierHallSmarts, khs.aaS KierHallSmarts, khs.aasC KierHallSmarts, khs.aaSe KierHallSmarts, khs.aasN KierHallSmarts, khs.dCH2 KierHallSmarts, khs.ddC KierHallSmarts, khs.ddsN KierHallSmarts, khs.ddssS KierHallSmarts, khs.ddssSe KierHallSmarts, khs.dNH KierHallSmarts, khs.dO KierHallSmarts, khs.dS KierHallSmarts, khs.dsCH KierHallSmarts, khs.dSe KierHallSmarts, khs.dsN KierHallSmarts, khs.dssC KierHallSmarts, khs.dssS KierHallSmarts, khs.dssSe KierHallSmarts, khs.dsssP KierHallSmarts, khs.sAsH2 KierHallSmarts, khs.sBr KierHallSmarts, khs.sCH3 KierHallSmarts, khs.sCl KierHallSmarts, khs.sF KierHallSmarts, khs.sGeH3 KierHallSmarts, khs.sI KierHallSmarts, khs.sLi KierHallSmarts, khs.sNH2 KierHallSmarts, khs.sNH3 KierHallSmarts, khs.sOH KierHallSmarts, khs.sPbH3 KierHallSmarts, khs.sPH2 KierHallSmarts, khs.ssAsH KierHallSmarts, khs.ssBe KierHallSmarts, khs.ssBH KierHallSmarts, khs.ssCH2 KierHallSmarts, khs.sSeH KierHallSmarts, khs.ssGeH2 KierHallSmarts, khs.sSH KierHallSmarts, khs.sSiH3 KierHallSmarts, khs.ssNH KierHallSmarts, khs.ssNH2 KierHallSmarts, khs.sSnH3 KierHallSmarts, khs.ssO KierHallSmarts, khs.ssPbH2 KierHallSmarts, khs.ssPH KierHallSmarts, khs.ssS KierHallSmarts, khs.sssAs KierHallSmarts, khs.sssB KierHallSmarts, khs.sssCH KierHallSmarts, khs.sssdAs KierHallSmarts, khs.ssSe KierHallSmarts, khs.sssGeH KierHallSmarts, khs.ssSiH2 KierHallSmarts, khs.sssN KierHallSmarts, khs.sssNH KierHallSmarts, khs.ssSnH2 KierHallSmarts, khs.sssP KierHallSmarts, khs.sssPbH KierHallSmarts, khs.ssssB KierHallSmarts, khs.ssssBe KierHallSmarts, khs.ssssC KierHallSmarts, khs.ssssGe KierHallSmarts, khs.sssSiH KierHallSmarts, khs.ssssN KierHallSmarts, khs.sssSnH KierHallSmarts, khs.ssssPb KierHallSmarts, khs.sssssAs KierHallSmarts, khs.ssssSi KierHallSmarts, khs.ssssSn KierHallSmarts, khs.sssssP KierHallSmarts, khs.tCH KierHallSmarts, khs.tN KierHallSmarts, khs.tsC KierHallSmarts, Kier1 kappa shape indices, Kier2 kappa shape indices, Kier3 kappa shape indices, largest chain, largest pi system, Mannhold LogP, MDEC-11 MDE, MDEC-12MDE, MDEC-13 MDE, MDEC-14MDE, MDEC-22 MDE, MDEC-23 MDE, MDEC-24 MDE, MDEC-33 MDE, MDEC-34 MDE, MDEC-44 MDE, MDEN-11 MDE, MDEN-12MDE, MDEN-13 MDE, MDEN-22 MDE, MDEN-23 MDE, MDEN-33 MDE, MDEO-11 MDE, MDEO-12MDE, MDEO-22 MDE, Petitjean number, rotatable bonds count, rule of five, topological surface area, vertex adjacency magnitude, weight, WPATH Wiener numbers, WPOL Wiener numbers, XLogP, and Zagreb index.

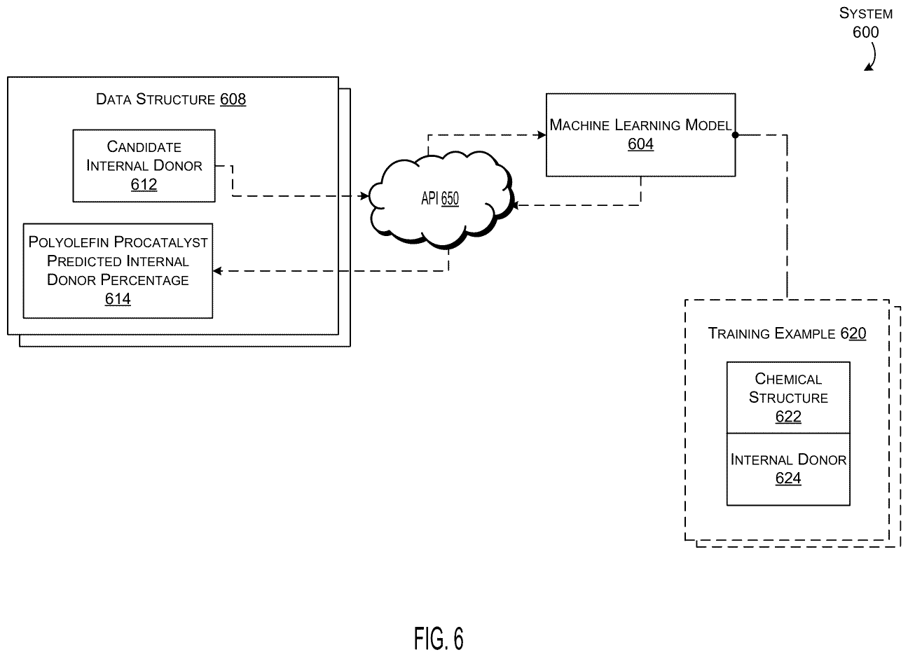

Referring to FIG. 6, illustrated is a block diagram of an example system 600 for predicting polyolefin procatalyst internal donor percentage using a machine learning model updated using a plurality of training examples, each training example including chemical structure and respective internal donor percentage.

Operations described in connection with FIG. 6 may be performed via one or more computing devices. For example, the computer system 900 of FIG. 9 may implement some or all of the operations described herein, through the execution of computer-readable instructions.

The system 600 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 600 may include one or more computing devices or servers that can perform various functions as described herein.

The system 600 may execute one or more machine learning models 604 in response to corresponding request(s) from an operator of the system 600 or from an external computing system. Outputs from the system 600 may be displayed on a display device of a computing device executing the system 600. For example, the computer system 900 of FIG. 9 may output predicted percent donor values and may display these values on display 914.

The system 600 may include one or more data structures 608, which may be stored in a storage device (e.g., storage device 910 of computer system 900 in FIG. 9), such as a solid-state device, magnetic disk, or optical disk for persistently storing information and instructions. The data structures 608 may be received from the storage device by a processor (e.g., processor 904 of computer system 900 in FIG. 9) via a bus (e.g., bus 902 of computer system 900 in FIG. 9).

The system 600 may include one or more machine learning models 604, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 600 may include one or more training examples 620, which may be stored in a storage device (e.g., storage device 910 of computer system 900 in FIG. 9), such as a solid-state device, magnetic disk, or optical disk for persistently storing information and instructions. The storage device in which the training examples 620 are stored may be the same or different than the storage device for storing the data structures 608. The training examples 620 may be received from the storage device by the processor (e.g., processor 904 of computer system 900 in FIG. 9) via the bus (e.g., bus 902 of computer system 900 in FIG. 9).

In some implementations, components of the system 600 may be implemented using multiple computing systems in communication with one other, for example, via a network. In some implementations, the system 600 may be implemented at least in part of a distributed computing system For example, the system 600 may be or may include an application server or webserver, which may include software modules to access or transmit data structures 608 stored by the memory in the system 600. In some implementations, one or more client devices of the system 600 may transmit data structures 608 to the distributed computing system in response to user input by an operator of the client devices. Such requests may include requests to initiating training and/or inference operations based on data submitted to the computing system maintaining the machine-learning model 604.

The system 600 may include a network to facilitate communication between remote components of the system 600 and/or between the system 600 and a remote computing system. The network may include any number of hardwired or wireless connections. Any or all of the computing devices described herein (e.g., the system 600) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 600) may also communicate wirelessly with other computing devices via a proxy device (e.g., a router, network switch, or gateway).

The system 600 may identify a data structure 608 comprising one or more chemical structures as candidate internal donors 612 for polyolefin procatalysts. The system may provide the data structure 608 as input to a model 604 to generate a predicted internal donor percentage for each resulting polyolefin procatalyst created using each chemical structure of the one or more chemical structures in the data structure 608. The system 600 may provide, for presentation, an output 614 including the predicted internal donor percentage for each of the one or more chemical structures for the internal donor.

The model 604 may be effective for operating on or generating data representative of chemical structures. For example, the candidate internal donor 612 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 600 includes one or more models 604. The models 604 may be trained using one or more training examples. The model 604 may include machine learning models or other models that can generate target outputs based on various types of inputs.

The system 600 may train, update, and/or configure one or more models 604. The model 604 may include machine learning models or other models that may generate target outputs based on various types of inputs. The system 600 may train, update, and/or configure the model 604 by modifying or updating one or more parameters responsive to evaluating one or more training examples 620 in view of expected or ground truth outputs.

The system 600 may include one or more application programming interfaces (APIs) 650, such as a distributed computing system (e.g., a cloud system) that stores data for the one or more models 604 described herein. In a non-limiting example, as input to the model 604, values from the data structure 608 (e.g., the candidate internal donor 612) may be an API call retrieved from the storage system as a response to a request transmitted using the API 650 of the storage system to the one or more models 604. As another example, the as input to the model 604, values from the training example 620 (e.g., chemical structure 622 and corresponding internal donor 624) may be retrieved from the storage system as a response to a request transmitted using the API 650 of the storage system to the one or more models 604.

The system 600 may operate on one or more candidate internal donors 612 (e.g., inference or runtime data; training data), which may be retrieved from one or more data structures 608. The data structure may include various databases, molecule editors, sensor data streams, user input sources, or a combination of any two or more thereof from which the system 600 can retrieve candidate internal donor 612 to process.

The data structure 608 may be maintained by one or more entities, which may be entities that maintain the system 600 and/or may be separate from entities that maintain the system 600. In some implementations, the system 600 uses data from different data sets, such as by using candidate internal donors 612 from a first data structure 608 to perform at least a first configuring (e.g., updating or training) of the model 604, and uses training data elements from a second data structure 608 to perform at least a second configuring of the models 604. For example, the first data structure 608 may include publicly available data, while the second data structure 608 can include domain-specific data (which may be limited in access as compared with the data of the first data structure 608). The system 600 may use candidate internal donors 612 from the same or different data structures 608 to configure, train, update, and/or perform runtime operations on candidate internal donors 612.

In some instances, a given candidate internal donor chemical structure 612 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 612 include SMILES representations, the system 600 may include all SMILES enumerations of the desired chemical species as input.

In addition to the internal donor chemical structure 612, the data structure 608 may include as input to the machine learning model 604 one or more additional parameters. These additional parameters may include one or more polymerization parameters, one or more polyolefin properties, or a combination thereof, as described herein.

The additional parameters may include a physical properties, a two-dimensional (2D) descriptor, a three-dimensional (3D) descriptor, or a combination of any two or morre thereof as described herein.

The output 614 from the machine learning model 604 may include a predicted internal donor percentage for the respective candidate internal donor 612. The predicted internal donor percentage may be a value or a range of values representing the relative amount of the candidate internal donor 612 that is added during preparation of the procatalyst.

In addition to or instead of predicted internal donor percentage, the output 614 may include one or more additional parameters. These additional parameters may include one or more polymerization parameters, one or more polyolefin properties, or a combination thereof, as described herein.

The training example 620 may include a chemical structure 622 and a corresponding internal donor percentage value 624. For example, the chemical structure 622 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 622 may be represented by multiple different data structures (e.g., different strings or enumerations). For example, multiple strings may be used to represent a single chemical species in order to improve the representation of that chemical species and describe the chemical species with all possible angles. Where the chemical structure 622 may include multiple strings that represent the same chemical species, the chemical structure may include some or all of the multiple strings that represent the same chemical species. For example, where the chemical structure 622 includes SMILES representations, the system 600 may include all SMILES enumerations of the desired chemical species as input.

The corresponding internal donor percentage value 624 may be a value or a range of values representing the relative amount of the internal donor represented by the chemical structure 622 that is added during preparation of the procatalyst.

In addition to the corresponding internal donor percentage value 624, the training example 620 may include as input to the machine learning model 604 one or more additional parameters. These additional parameters may include one or more polymerization parameters, one or more polyolefin properties, or a combination thereof, as described herein. The additional parameters may include a physical properties, a two-dimensional (2D) descriptor, a three-dimensional (3D) descriptor, or a combination of any two or more thereof as described herein.

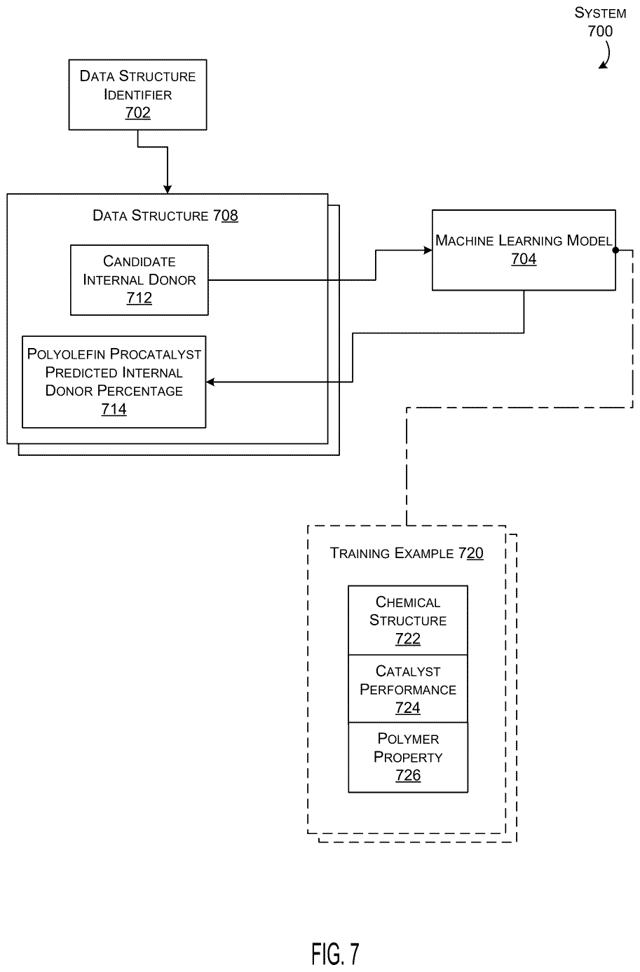

Referring to FIG. 7, illustrated is a block diagram of an example system 700 for predicting polyolefin procatalyst internal donor percentage 714 using a machine learning model updated using a plurality of training examples, each training example 720 including chemical structure and respective catalyst performance 724 and polymer property 726.

Operations described in connection with FIG. 7 may be performed via one or more computing devices. For example, the computer system 900 of FIG. 9 may implement some or all of the operations described herein, through the execution of computer-readable instructions.

The system 700 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include a storage device) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The system 700 may include one or more computing devices or servers that can perform various functions as described herein.

The system 700 may execute one or more machine learning models 704 in response to corresponding request(s) from an operator of the system 700 or from an external computing system. Outputs from the system 700 may be displayed on a display device of a computing device executing the system 700. For example, the computer system 900 of FIG. 9 may output predicted percent donor values and may display these values on display 914.

The system 700 may include one or more data structures 708, which may be stored in a storage device (e.g., storage device 910 of computer system 900 in FIG. 9), such as a solid-state device, magnetic disk, or optical disk for persistently storing information and instructions. The data structures 708 may be received from the storage device by a processor (e.g., processor 904 of computer system 900 in FIG. 9) via a bus (e.g., bus 902 of computer system 900 in FIG. 9).

The system 700 may include one or more machine learning models 704, which may be stored in a main memory (e.g., main memory 906 of computer system 900 in FIG. 9) and received by the processor (e.g., processor 904 of computer system 900 in FIG. 9) via the bus (e.g., bus 902 of computer system 900 in FIG. 9).

The system 700 may include one or more training examples 720, which may be stored in a storage device (e.g., storage device 910 of computer system 900 in FIG. 9), such as a solid-state device, magnetic disk, or optical disk for persistently storing information and instructions. The storage device in which the training examples 720 are stored may be the same or different than the storage device for storing the data structures 708. The training examples 720 may be received from the storage device by the processor (e.g., processor 904 of computer system 900 in FIG. 9) via the bus (e.g., bus 902 of computer system 900 in FIG. 9).

In some implementations, components of the system 700 may be implemented using multiple computing systems in communication with one other, for example, via a network. In some implementations, the system 700 may be implemented at least in part of a distributed computing system For example, the system 700 may be or may include an application server or webserver, which may include software modules to access or transmit data structures 708 stored by the memory in the system 700. In some implementations, one or more client devices of the system 700 may transmit data structures 708 to the distributed computing system in response to user input by an operator of the client devices. Such requests may include requests to initiating training and/or inference operations based on data submitted to the computing system maintaining the machine-learning model 704.

The system 700 may include a network to facilitate communication between remote components of the system 700 and/or between the system 700 and a remote computing system. The network may include any number of hardwired or wireless connections. Any or all of the computing devices described herein (e.g., the system 700) may communicate wirelessly (e.g., via Wi-Fi, cellular communication, radio, etc.) with a transceiver that is hardwired (e.g., via a fiber optic cable, a CAT5 cable, etc.) to other computing devices. Any or all of the computing devices described herein (e.g., the system 700) may also communicate wirelessly with other computing devices via a proxy device (e.g., a router, network switch, or gateway).

The system 700 may include a data structure identifier 702 to identify a data structure comprising one or more chemical structures as candidate internal donors 712 for polyolefin procatalysts. The data structure identifier 702 may identify a data structure 708 comprising one or more chemical structures as candidate internal donors 712 for polyolefin procatalysts. The system may provide the data structure 708 as input to a model 704 to generate a predicted internal donor percentage for each resulting polyolefin procatalyst created using each chemical structure of the one or more chemical structures in the data structure 708. The system 700 may provide, for presentation, an output 714 including the predicted internal donor percentage for each of the one or more chemical structures for the internal donor.

The model 704 may be effective for operating on or generating data representative of chemical structures. For example, the candidate internal donor 712 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof. For example, SMILES is a form of line notation for describing the structure of chemical species using short ASCII strings.

The system 700 includes one or more models 704. The models 704 may be trained using one or more training examples. The model 704 may include machine learning models or other models that can generate target outputs based on various types of inputs.

The system 700 may train, update, and/or configure one or more models 704. The model 704 may include machine learning models or other models that may generate target outputs based on various types of inputs. The system 700 may train, update, and/or configure the model 704 by modifying or updating one or more parameters responsive to evaluating one or more training examples 720 in view of expected or ground truth outputs.

The data structure 708 may be maintained by one or more entities, which may be entities that maintain the system 700 and/or may be separate from entities that maintain the system 700. In some implementations, the system 700 uses data from different data sets, such as by using candidate internal donors 712 from a first data structure 708 to perform at least a first configuring (e.g., updating or training) of the model 704, and uses training data elements from a second data structure 708 to perform at least a second configuring of the models 704. For example, the first data structure 708 may include publicly available data, while the second data structure 708 can include domain-specific data (which may be limited in access as compared with the data of the first data structure 708). The system 700 may use candidate internal donors 712 from the same or different data structures 708 to configure, train, update, and/or perform runtime operations on candidate internal donors 712.

In some instances, a given candidate internal donor chemical structure 712 may be represented by multiple different data structures (e.g., different strings or enumerations). For example, multiple strings may be used to represent a single chemical species in order to improve the representation of that chemical species and describe the chemical species with all possible angles. Where the candidate internal donor chemical structure can include multiple strings that represent the same chemical species, the chemical structure may include some or all of the multiple strings that represent the same chemical species. For example, where the candidate internal donor 712 include SMILES representations, the system 700 may include all SMILES enumerations of the desired chemical species as input.

In addition to the internal donor chemical structure 712, the data structure 708 may include as input to the machine learning model 704 one or more additional parameters. These additional parameters may include one or more polymerization parameters, one or more polyolefin properties, or a combination thereof, as described herein.

The additional parameters may include a physical properties, a two-dimensional (2D) descriptor, a three-dimensional (3D) descriptor, or a combination of any two or more thereof as described herein.

The output 714 from the machine learning model 704 may include a predicted internal donor percentage for the respective candidate internal donor 712. The predicted internal donor percentage may be a value or a range of values representing the relative amount of the candidate internal donor 712 that is added during preparation of the procatalyst.

In addition to or instead of predicted internal donor percentage, the output 714 may include one or more additional parameters. These additional parameters may include one or more polymerization parameters, one or more polyolefin properties, or a combination thereof, as described herein.

The training example 720 may include a chemical structure 722 and a corresponding catalyst performance 724. For example, the chemical structure 722 may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

In some instances, a given chemical structure 722 may be represented by multiple different data structures (e.g., different strings or enumerations). For example, multiple strings may be used to represent a single chemical species in order to improve the representation of that chemical species and describe the chemical species with all possible angles. Where the chemical structure 722 may include multiple strings that represent the same chemical species, the chemical structure may include some or all of the multiple strings that represent the same chemical species. For example, where the chemical structure 722 includes SMILES representations, the system 700 may include all SMILES enumerations of the desired chemical species as input.

The corresponding catalyst performance 724 may be a value or a range of values representing the efficiency and/or effectiveness of the catalyst in facilitating the polyolefin reaction. Catalyst performance 724 may include one or more polymerization parameters, which may include one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

The corresponding polymer property 726 may include one or more polyolefin properties. The one or more polyolefin properties may include one or more of a xylene solubility (XS) value, a polydispersity index (PDI) value, or melt flow response (MFR) value.

In addition to the corresponding catalyst performance 724 and polymer property 726, the training example 720 may include as input to the machine learning model 704 one or more additional parameters. These additional parameters may include one or more polymerization parameters, one or more polyolefin properties, or a combination thereof, as described herein. The additional parameters may include a physical properties, a two-dimensional (2D) descriptor, a three-dimensional (3D) descriptor, or a combination of any two or more thereof as described herein.

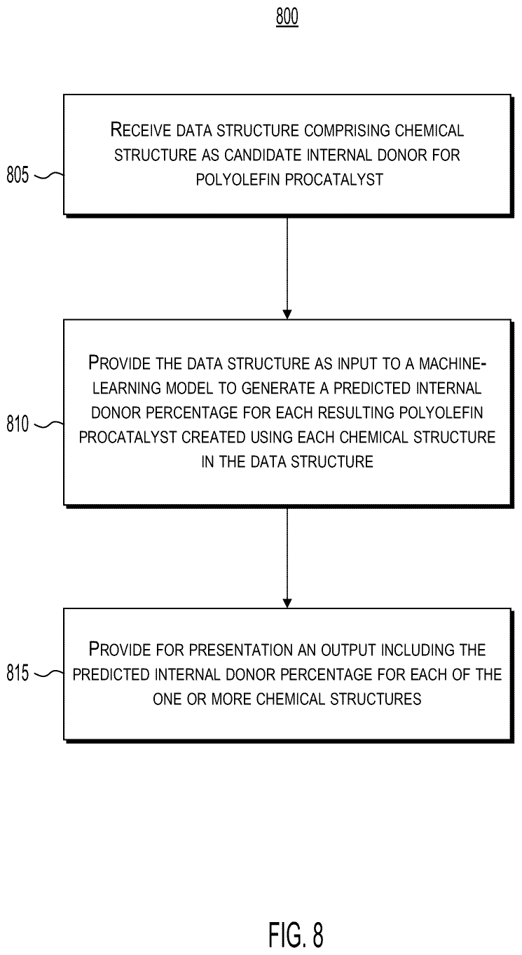

FIG. 8 illustrates a flow chart of an example method 800 for determining a predicting internal donor percentage for corresponding chemical structure for a candidate internal donor in polyolefin procatalyst. The method 800 may be performed by the system 100, the system 200, the system 300, the system 400, the system 500, the system 600, or the system 700. The method 800 may be performed, at least in part, for example, by the model 104, or any systems described herein. It should be understood that the method 800 shown in FIG. 8 is an example, and that additional steps may be performed, steps may be omitted, or steps may be performed in a different order than shown, to achieve desired results.

At act 805 of method 800, the system receives a data structure including the chemical structure as a candidate internal donor for a polyolefin procatalyst. For example, the system may identify a data structure comprising one or more chemical structures as candidate internal donors for polyolefin procatalysts. For example, the candidate internal donor may include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, or a combination of any two or more thereof.

At act 810, the system provides the data structure as input to a machine learning model to generate a predicted internal donor percentage for each resulting polyolefin procatalyst created using each chemical structure in the data structure. The machine learning model may be trained using training examples as disclosed herein.

At act 815, the system provides for presentation an output including the predicted internal donor percentage for each of the one or more chemical structures. The output may include one or more additional parameters. These additional parameters may include one or more polymerization parameters, one or more polyolefin properties, or a combination thereof, as disclosed herein.

FIG. 9 is a component diagram of an example computing system suitable for use in the various implementations described herein, according to an example implementation. For example, the computing system 900 may implement any of the systems disclosed herein, or various other example systems and devices described in the present disclosure.

The computing system 900 includes a bus 902 or other communication component for communicating information and a processor 904 coupled to the bus 902 for processing information. The computing system 900 also includes main memory 906, such as a RAM or other dynamic storage device, coupled to the bus 902 for storing information, and instructions to be executed by the processor 904. Main memory 906 can also be used for storing position information, temporary variables, or other intermediate information during execution of instructions by the processor 904. The computing system 900 may further include a ROM 908 or other static storage device coupled to the bus 902 for storing static information and instructions for the processor 904. A storage device 910, such as a solid-state device, magnetic disk, or optical disk, is coupled to the bus 902 for persistently storing information and instructions.

The computing system 900 may be coupled via the bus 902 to a display 914, such as a liquid crystal display, or active matrix display, for displaying information and notifications to a user. An input device 912, such as a keyboard including alphanumeric and other keys, may be coupled to the bus 902 for communicating information, and command selections to the processor 904. In another implementation, the input device 912 has a touch screen display. The input device 912 can include any type of biometric sensor, or a cursor control, such as a mouse, a trackball, or cursor direction keys, for communicating direction information and command selections to the processor 904 and for controlling cursor movement on the display 914.

In some implementations, the computing system 900 may include a communications adapter 916, such as a networking adapter. Communications adapter 916 may be coupled to bus 902 and may be configured to enable communications with a computing or communications network or other computing systems. In various illustrative implementations, any type of networking configuration may be achieved using communications adapter 916, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein can be achieved by the computing system 900 in response to the processor 904 executing an implementation of instructions contained in main memory 906. Such instructions can be read into main memory 906 from another computer-readable medium, such as the storage device 910. Execution of the implementation of instructions contained in main memory 906 causes the computing system 900 to perform the illustrative processes described herein. One or more processors in a multi-processing implementation may also be employed to execute the instructions contained in main memory 906. In alternative implementations, hard-wired circuitry may be used in place of or in combination with software instructions to implement illustrative implementations. Thus, implementations are not limited to any specific combination of hardware circuitry and software.

The present technology, thus generally described, will be understood more readily by reference to the following examples, which are provided by way of illustration and are not intended to be limiting of the present technology.

### EXAMPLES

Prior attempts at predicting internal donor percentage using a trained machine learning model using data including many different parameters was unsuccessful. It was suspected that certain subsets of data could be negatively skewing data outputs. Therefore, a smaller subset of data with fewer inputs was chosen to prioritize fewer parameters. The system of FIG. 1 was used to train the machine learning model and predict internal donor percentage.

Donor incorporation percentage was chosen as the priority of interest, since there may be a high correlation with a candidate donor's ability to coordinate and the chance for a successful procatalyst.

The machine learning model was trained using training examples including SMILES chemical structures and corresponding internal donor percentage. A set of 5 data structures including 39 chemical structures were excluded from the training examples to use as a validation set.

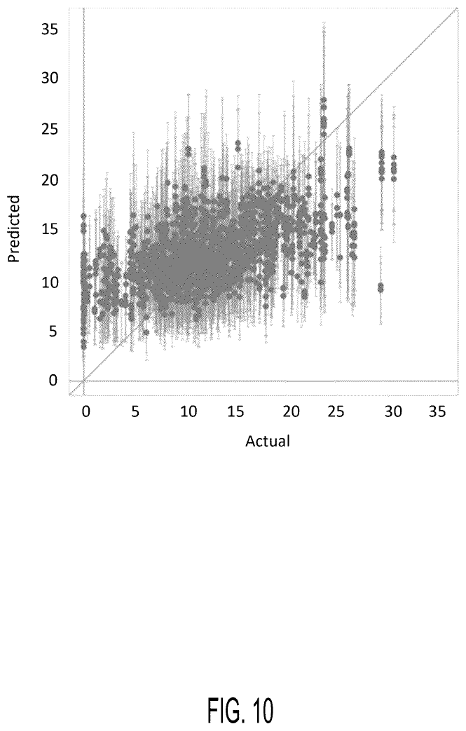

The model was analyzed and feature importance was reviewed. Model outputs for internal donor percentage were consistent with known chemistry principles. FIG. 10 is a graph of actual vs. predicted internal donor percentage for respective candidate internal donors generated using the system of FIG. 1, indicating good alignment.

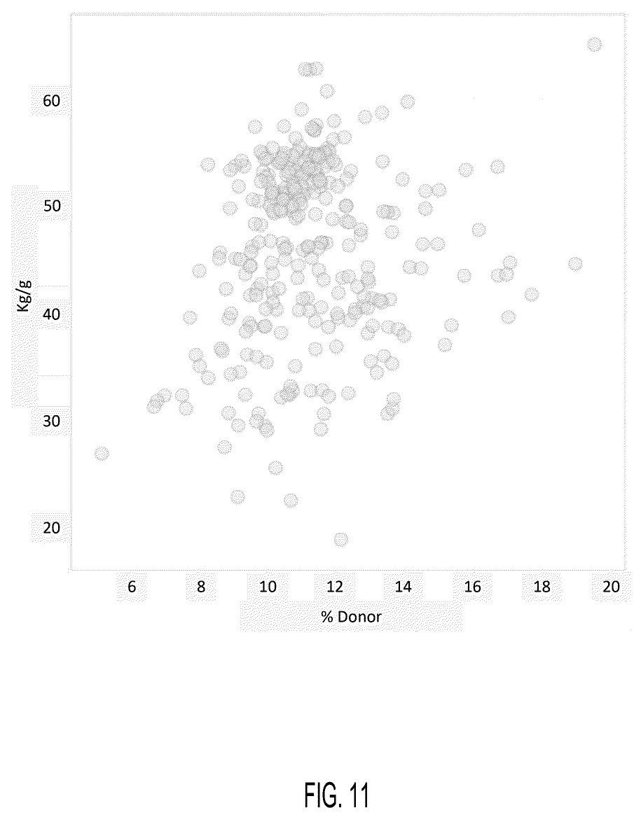

Further refinement was performed on the model so that large queries of molecules could be tested against the model with one input: FIG. 11 is a graph of internal donor percentage (“% donor”) vs. catalyst activity (kg/g) for candidate internal donors generated using the system of FIG. 1, indicating a positive correlation.

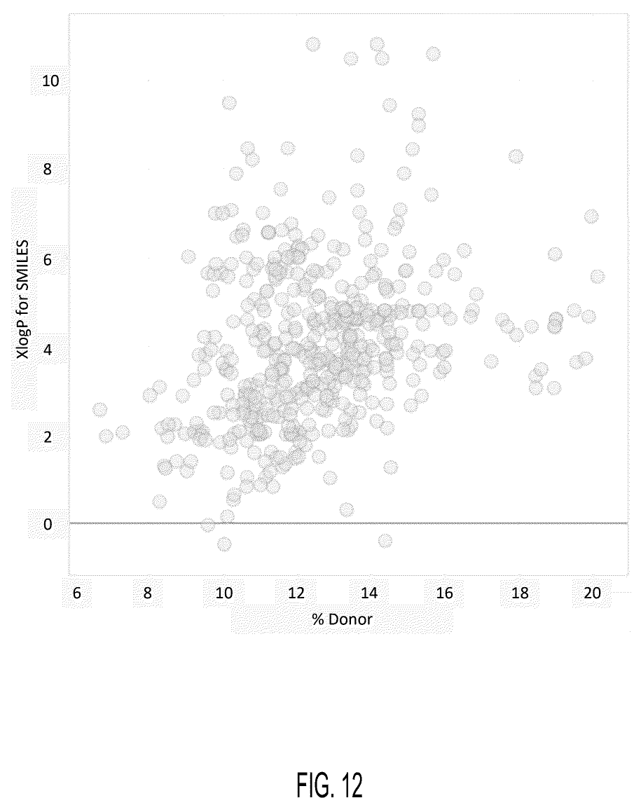

Model validation was further performed by using 5 excluded data structures as input to the model. FIG. 12 is a graph of internal donor percentage vs. octanol/water partition coefficient (“XLogP”) for candidate internal donors generated using the system of FIG. 1.

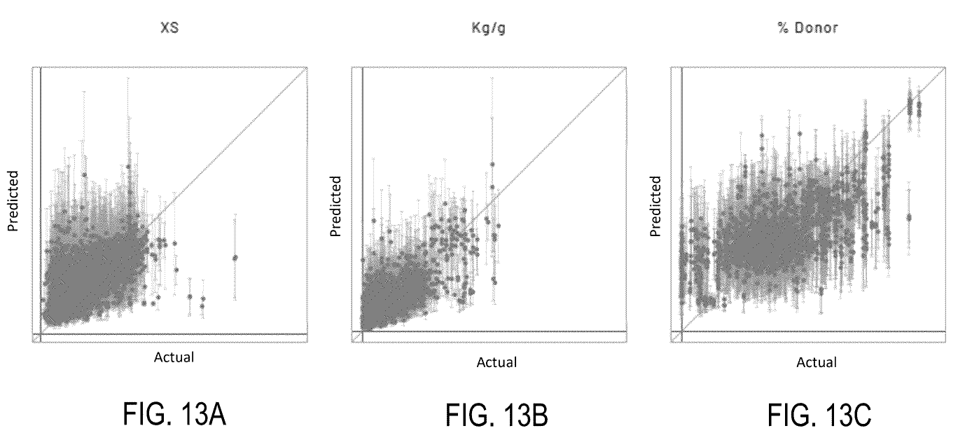

In another example, training examples input to the model for training included internal donor percentage and additional parameters. These additional parameters included XS and catalytic activity, in accordance with the system in FIG. 2. Data structures were input to the model trained with these additional parameters to predict XS, catalytic activity, and internal donor percentage. FIGS. 13A-13C are graphs of actual vs. predicted parameters for respective candidate internal donors generated using the system of FIG. 2. FIG. 13A is a graph of actual vs. predicted xylene solubility (XS). FIG. 13B is a graph of actual vs. predicted catalytic activity. FIG. 13C is a graph of actual vs. predicted internal donor percentage.

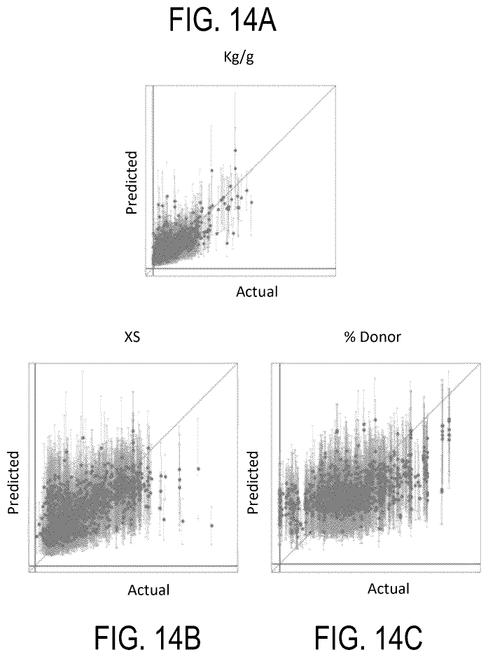

In another example, training examples input to the model for training included internal donor percentage and physical properties, including DFT calculations, in accordance with the system in FIG. 3. FIGS. 14A-14E are graphs of predicted vs. actual parameters for respective candidate internal donors generated using the system of FIG. 3. FIG. 14A is a graph of actual vs. predicted catalytic activity. FIG. 14B is a graph of actual vs. predicted xylene solubility. FIG. 14C is a graph of actual vs. predicted internal donor percentage.

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

The implementations described herein have been described with reference to drawings. The drawings illustrate certain details of specific implementations that implement the systems, methods, and programs described herein. However, describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As understood by one of ordinary skill in the art, “internal donor percentage”, “internal donor percentage in the procatalyst composition”, and “internal donor incorporation in polyolefin procatalyst percentage” refer to the relative amount of the internal donor incorporated into the Ziegler-Natta type procatalyst. The internal donor percentage may be the percent of donor remaining in procatalyst composition after preparation of the procatalyst. The internal donor percentage may be defined as the weight percentage of the internal donor relative to the weight of all other catalyst components in the procatalyst composition. “Internal electron donor”, “internal donor,” and “donor” may have the same meaning and may be used interchangeably.

As understood by one of ordinary skill in the art, “xylene soluble” (also referred to as “XS”) may be defined as the weight percent of resin that remains in solution after a sample of polypropylene random copolymer resin is dissolved in hot xylene and the solution is allowed to cool to 25 °C. This is also referred to as the gravimetric XS method according to ASTM D5492-98 using a 90 minute precipitation time and is a referred to herein as the “wet method.” Xylene solubles is measured on the reactor powder without the addition of any other additives.

The xylene soluble portion is determined by a method adapted from ASTM D5492-06 and also sometimes referred to herein as the “wet method”. The procedure consists of weighing 2 g of sample and dissolving the sample in 200 mL o-xylene in a 400 ml flask with 24/40 joint. The flask is connected to a water cooled condenser and the contents are stirred and heated to reflux under nitrogen (N₂), and then maintained at reflux for an additional 30 minutes. The solution is then cooled in a temperature controlled water bath at 25° C. for 90 minutes to allow the crystallization of the xylene insoluble fraction. Once the solution is cooled and the insoluble fraction precipitates from the solution, the separation of the xylene soluble portion (XS) from the xylene insoluble portion (XI) is achieved by filtering through 25 micron filter paper. One hundred mL of the filtrate is collected into a pre-weighed aluminum pan, and the o-xylene is evaporated from this 100 mL of filtrate under a nitrogen stream. Once the solvent evaporated from this 100 mL of filtrate under a nitrogen stream. Once the solvent is evaporated, the pan and contents are placed in a 100° C. vacuum oven for 30 minutes or until dry. The pan is then allowed to cool to room temperature and weighed. The xylene soluble portion is calculated as XS (wt. %)=[(m₃−m₂)×2/mL]×100, where mL is the original weight of the sample used, m₂ is the weight of empty aluminum pan, and m₃ is the weight of the pan and residue.

As understood by one of ordinary skill in the art, “H₂ charge” and “H₂ charge value” refer to the amount of hydrogen gas added during the polymerization process of the polyolefin to control the molecular weight of the resulting polyolefin by acting as a chain transfer agent. The H₂ charge value may influence the length of the polyolefin chain to provide precise control of one or more polymer properties.

As understood by one of ordinary skill in the art, “catalytic activity” and catalytic activity value” refer to the ability of the catalyst to accelerate the polyolefin polymerization reaction. The catalytic activity may be expressed as, for example, kg of polymer produced per g of catalyst, or catalytic efficiency (CE) in units of kg of polymer per gram of catalyst during the polymerization for 1 hour.

As understood by one of ordinary skill in the art, “catalyst lifetime” and “catalyst lifetime value” refer to the period during which the catalyst remains effective in facilitating the polymerization reaction before it loses its activity or requires replacement or regeneration. A longer catalyst lifetime may be desirable in industrial processes, as it reduces the frequency of catalyst replacement, lowers costs, and ensures consistent product quality.

As understood by one of ordinary skill in the art, “polydispersity index” (also referred to as PDI) is a measure of the distribution of molecular weight in a given polyolefin sample, and may be calculated as the ratio of the weight average molecular weight to the number average molecular weight.

As understood by one of ordinary skill in the art, “melt flow rate” (also referred to as MFR, melt flow index, and MFI) is a measure of the flowability of the polyolefin melt under a temperature and a load condition. MFR may be used to assess the viscosity and processability of polyolefins. The MFR is measured according to ASTM standard D1238 T. The value of MFR may be given in units of grams per 10 minutes (g/ 10 min), which represents the mass of polymer extruded through a standard die over a 10-minute period under specified conditions.

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, for example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, for example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term-for example, “about 10 wt. %” would be understood to mean “9 wt. % to 11 wt. %.” It is to be understood that when “about” precedes a term, the term is to be construed as disclosing “about” the term as well as the term without modification by “about” for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof-for example, “A, B, and/or C” would mean “A or B or C; A and B; A and C; B and C; or the combination of A, B, and C.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A system, comprising:
one or more processors coupled to non-transitory memory, the one or more processors configured to:
identify a data structure comprising one or more chemical structures as candidate internal donors for polyolefin procatalysts, the one or more chemical structures represented as a data string defined according to a predetermined specification;
provide the data structure as input to a machine-learning model to generate a predicted internal donor percentage for each resulting polyolefin procatalyst created using each chemical structure of the one or more chemical structures in the data structure,
wherein the machine-learning model is updated using a plurality of training examples, each of the plurality of training examples comprising:
(i) a respective chemical structure for each candidate internal donor, and, optionally,
(ii) a respective internal donor incorporation in polyolefin procatalyst percentage corresponding to the respective chemical structure; and
provide, for presentation, an output including the predicted internal donor percentage for each of the one or more chemical structures for internal donor.

2. The system of claim 1, wherein the one or more processors are further configured to update the machine-learning model using the plurality of training examples.

3. The system of claim 2, wherein each of the plurality of training examples further comprises one or more polymerization parameters and one or more polyolefin properties.

4. The system of claim 3, wherein the one or more polymerization parameters comprise one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or melt flow response (MFR) value.

5. The system of claim 2, wherein each of the plurality of training examples further comprises one or more of (i) a set of calculated physical properties having at least one of an electronegativity value, a hardness value, or a dipole moment, and (ii) a set of calculated two-dimensional (2D) and/or three-dimensional (3D) descriptors.

6. The system of claim 1, wherein the one or more processors are further configured to:
generate a feature importance for one or more values of a dataset; and
generate the plurality of training examples based on the feature importance.

7. The system of claim 1, wherein the one or more processors are further configured to:
generate, for each chemical structure of the one or more chemical structures, a respective set of molecular descriptors based on the data string of the chemical structure; and
provide the respective set of molecular descriptors as input to the machine-learning model.

8. The system of claim 1, wherein the one or more processors are further configured to provide the data structure as input to using at least one application programming interface (API) call.

9. The system of claim 1, wherein the one or more processors are further configured to generate, using the machine-learning model for each chemical structure of the one or more chemical structures, a confidence score indicating a likelihood that the predicted internal donor percentage for the chemical structure is accurate.

10. The system of claim 1, wherein the one or more processors are further configured to generate one or more plots based on the output of the machine-learning model.

11. A method, comprising:
receiving, by one or more processors coupled to non-transitory memory, a data structure comprising one or more chemical structures as candidate internal donors in polyolefin procatalyst, the one or more chemical structures represented as a data string defined according to a predetermined specification;
providing, by the one or more processors, the data structure as input to a machine-learning model to generate a predicted internal donor percentage for each resulting polyolefin procatalyst created using each chemical structure of the one or more chemical structures in the data structure,
wherein the machine-learning model is updated using a plurality of training examples, each of the plurality of training examples comprising:
(i) a respective chemical structure, and
(ii) a respective internal donor percentage corresponding to the respective chemical structure; and
providing, by the one or more processors, for presentation an output including the predicted internal donor percentage for each of the one or more chemical structures.

12. The method of claim 11, further comprising updating, by the one or more processors, the machine-learning model using the plurality of training examples.

13. The method of claim 12, wherein each of the plurality of training examples comprise one or more polymerization parameters and one or more polyolefin properties.

14. The method of claim 13, wherein the one or more polymerization parameters comprise one or more of an H₂ charge value, an external donor structure, a titanium (Ti) to external donor ratio, a catalytic activity value, a co-catalyst structure, a ratio of aluminum (Al) to Ti, a catalyst lifetime value, or a polymer bulk density value, and wherein the one or more polyolefin properties comprise one or more of a xylene solubility (XS) value, a polydispersity index (PDI) value, or a melt flow response (MFR) value.

15. The method of claim 12, wherein each of the plurality of training examples comprises one or more of a set of calculated physical properties, a set of calculated two-dimensional (2D) and/or three-dimensional (3D) descriptors, an electronegativity value, a hardness value, or a dipole moment.

16. The method of claim 11, further comprising:
generating, by the one or more processors, a feature importance for one or more values of a dataset; and
generating, by the one or more processors, the plurality of training examples based on the feature importance.

17. The method of claim 11, further comprising:
generating, by the one or more processors, for each chemical structure of the one or more chemical structures, a respective set of molecular descriptors based on the data string of the chemical structure; and
providing, by the one or more processors, the respective set of molecular descriptors as input to the machine-learning model.

18. A system, comprising:
one or more processors coupled to non-transitory memory, the one or more processors configured to:
identify a data structure comprising one or more chemical structures as candidate internal donors for polyolefin procatalysts, the one or more chemical structures represented as a data string defined according to a predetermined specification;
provide the data structure as input to a machine-learning model to generate a predicted polymer performance and a predicted catalyst performance for each resulting polyolefin procatalyst created using each chemical structure of the one or more chemical structures in the data structure,
wherein the machine-learning model is updated using a plurality of training examples, each of the plurality of training examples comprising:
(i) a respective chemical structure,
(ii) a respective catalyst performance, and
(iii) one or more respective polymer properties
corresponding to the respective chemical structure; and
provide, for presentation, an output including the predicted polymer performance and a predicted catalyst performance for each of the one or more chemical structures.

19. The system of claim 18, wherein the respective catalyst performance comprises one or more of a catalytic activity value or a catalyst lifetime value, and wherein the one or more respective polymer properties comprise one or more of a xylene solubility (XS) value, a polydispersity index (PDI) value, or a melt flow response (MFR) value.

20. The system of claim 18, wherein the one or more processors are further configured to generate, using the machine-learning model for each chemical structure of the one or more chemical structures, one or more confidence scores indicating a likelihood that the predicted polymer performance and the predicted catalyst performance are accurate.
